(19) United States
(12) Patent Application Publication
(10) Pub. No.: US 2023/0094369 A1
(43) Pub. Date: Mar. 30, 2023

(54) SYNTHESIS OF GLYCOSYLATED SPHINGOID BASES OF INTEREST OR ANALOGUES THEREOF

(71) Applicant: CarboCode S.A., Cantanhede (PT)

(72) Inventors: Ferenc Horvath, Pilisszentkereszt (HU); Györgyi Osztrovszky, Kisvarda (HU); Gyula Dekany, Queensland (AU); Agathe Bronikowski, Adelsheim (DE); Piroska Kovacs-Penzes, Jaszbereny (HU); Rafael Soares, Tamengos (PT); Jorge Santos, Alenquer (PT); Fabio Pereira, Cacém (PT); Osama Mahmoud, Konstanz (DE); Nagy Csaba, Konstanz (DE); Dário Jorge Silva Neves, Louriçal (PT)

(21) Appl. No.: 17/801,642

(22) PCT Filed: Feb. 24, 2021
(86) PCT No.: PCT/EP2021/054516

§ 371 Date: Aug. 23, 2022

(30) Foreign Application Priority Data
Feb. 24, 2020 (CH) 00219/20

(51) Int. Cl. C12P 19/44; C12P 19/18

(52) U.S. Cl. CPC C12P 19/44; C12P 19/18

(57) ABSTRACT

The present invention relates to a method for producing a glycosylated sphingoid base of interest or an analogue thereof, the method comprising providing an internalized exogenous precursor and a genetically modified cell, wherein one or more glycosylation reactions can be performed on the exogenous precursor in the genetically modified cell, the genetically modified cell comprising one or more nucleic acid sequences encoding one or more glycosyltransferase enzymes.

### FIELD OF THE INVENTION

The present invention relates to a method for producing glycosylated sphingoid bases of interest or analogues thereof using a biotechnological approach.

### BACKGROUND OF THE INVENTION

Glycosphingolipids (GSLs) are a class of glycolipids mainly found on the surface of eukaryotic cells. Their structure consists of a glycan moiety conjugated to a sphingolipid unit (ceramide). Owing to the diversity of the glycan moiety, GSLs represent a large family of glycoconjugates and to date more than 300 different structures have been identified.

GSLs are involved in diverse biological processes and play important structural and functional roles. For instance, they contribute to cell-cell recognition, communication, and intercellular adhesion (S.-I. Hakomori, Glycoconjugate J. 2001, 143-151). They have been shown to be involved in diverse immune processes (T. Zhang, A. A. de Waard, M. Wuhrer, R. M. Spaapen, Front. Immunol. 2019) as well as cancer angiogenesis and progression (S. Groux-Degroote, Y. Guerardel, P. Delannoy, Chem. Bio. Chem. 2017, 1146-1154). Further more, certain GSLs are found in brain and play roles in neurological diseases (Kolter, ISRN Biochem. 2012).

GSLs hold great potential as therapeutics and as tools for the study of important biological processes, however they are not readily available for fundamental and clinical research. In fact, GSLs are characterized by a high structural complexity and their preparation represents a challenge.

Processes for the preparation of GSLs that are based on chemical and enzymatic synthesis exist. However, these approaches possess several limitations.

Chemical synthesis (J. A. Morales-Serna, O. Boutureira, Y. Diaz, M. I. Matheu, S. Castillon, Carbohydr. Res. 2007) is usually performed in two steps. First, the glycan moiety is synthesised and then coupled to ceramide or a sphingoid base. Drawbacks connected to this approach are the control of stereo- and regiochemistry, the need of multiple protecting group manipulations, difficult purification and scale-up.

Alternatively, GSLs can be obtained via an enzymatic approach where a sphingoid base or a glycosylated sphingoid base is elongated via sequential glycosylation catalysed by glycosyltransferases (GTs). The final GSL is then obtained by coupling the lyso-form to a fatty acid (WO 99/28491 A1). Limitations to this approach include engineering the expression and isolating the pure enzyme and the use of expensive glycosyl nucleotide donors.

### SUMMARY OF THE INVENTION

The present inventors have found that surprisingly, exogenous precursors of General Formula I can be internalized by a cell, where the precursors can be subjected to glycosylation reactions. The inventors accordingly established for the first time a biotechnological route for producing complex glycosylated sphingoid bases or analogues thereof.
(1) The present invention relates to a method for producing a glycosylated sphingoid base of interest or an analogue thereof, the method comprising the steps of:
   a) Providing an exogenous precursor and a genetically modified cell, wherein one or more glycosylation reactions can be performed on the exogenous precursor or on the glycosylated derivative thereof in the genetically modified cell, the genetically modified cell comprising one or more nucleic acid sequences encoding one or more glycosyltransferases, and wherein the exogenous precursor is a compound of General Formula I

Y—X—R General Formula I,
wherein
Y is a glycosyl moiety,
X is O, S, NH or CH₂, linking Y to R by an O-, S-, N- or C-glycosidic linkage, respectively, wherein the glycosidic linkage is preferably a beta-glycosidic linkage,
and R is a group of General Formula IIa or General Formula IIb:

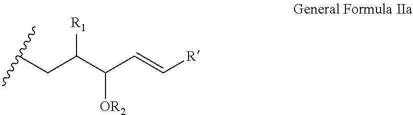

wherein
R′ is H, aryl or an alkyl chain having 1-43 carbon atoms, which may be a straight chain or branched, and/or which may be saturated or contain one or more double and/or triple bonds, and/or which may contain one or more functional groups, the functional group being preferably selected from the group consisting of a hydroxyl group, an alkoxy group, an acyloxy group, a primary, secondary or tertiary amine, an acylamido group, a thiol group, a thioether or a phosphorus-containing functional group,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl;

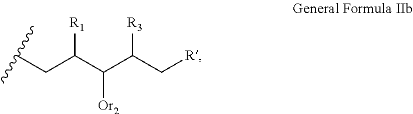

wherein R′, R₁, R₂, are as defined in General Formula IIa, and
R₃ is H, OH or OR₆, wherein R₆ is selected from substituted or unsubstituted alkyl or substituted or unsubstituted acyl;
b) Culturing said genetically modified cell in a culture medium comprising said exogenous precursor, whereby
   i. the exogenous precursor is internalized by the cell, and
   ii. one or more glycosylation reactions are performed on the internalized exogenous precursor or on a glycosylated derivative thereof by the one or more glycosyltransferases, to form the glycosylated sphingoid base of interest,
c) Optionally isolating the glycosylated sphingoid base of interest or the analogue thereof characterized by General Formula III from the genetically modified cell and/or from the culture medium.
(2) The method according to (1), wherein the genetically modified cell is a yeast cell or a bacterial cell, preferably an E. coli cell.
(3) The method according to (1) or (2), wherein the one or more glycosyltransferase enzymes comprise one or more sialyltransferases and/or one or more fucosyltransferases, especially one or more sialyltransferases.
(4) The method according to (1) or (2), wherein the one or more glycosyltransferase enzymes are selected from the group consisting of β-1,3-N-acetylglucosaminyltransferase, β-1,6-N-acetylglucosaminyltransferase, β-1,3-galactosyltransferase, β-1,4-galactosyltransferase, β-1,4-N-acetylgalactosaminyltransferase, β-1,3-N-acetylgalactosaminyltransferase, β-1,3-glucoronosyltransferase, α-2,3-sialyltransferase, α-2,6-sialyltransferase, α-2,8-sialyltransferase, α-1,2-fucosyltransferase, α-1,3-fucosyltransferase, α-1,4-fucosyltransferase, α-1,4-galactosyltransferase, α-1,3-galactosyltransferase or a combination thereof.
(5) The method according to any one of (1) to (4), wherein Y of General Formula I is a monosaccharide moiety, a disaccharide moiety or a trisaccharide moiety, preferably a monosaccharide moiety or a disaccharide moiety.
(6) The method according to any one of (1) to (4), wherein the exogenous precursor is a compound of General Formula Ia:

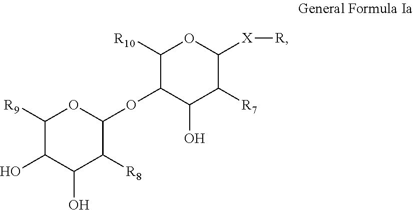

wherein
X and R are as defined for General Formula I in (1),
R₇ and R₈ are independently selected from the group consisting of OH, NH₂ and NH-acyl,
and
R₉ and R₁₀ are independently selected from the group consisting of —CH₂—OH and an C₁₋₆ alkyl, preferably methyl.
(7) The method according to any one of (1) to (4), wherein the exogenous precursor is a compound of General Formula Ib:

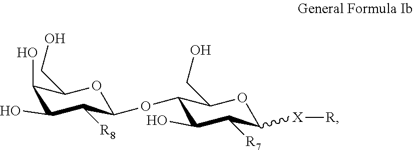

wherein
X and R are as defined for General Formula I in (1)
and
R₇ and R₈ are as defined for General Formula Ia in (6).
(8) The method according to any one of (1) to (7), wherein the exogenous precursor is a compound of General Formula Ic:

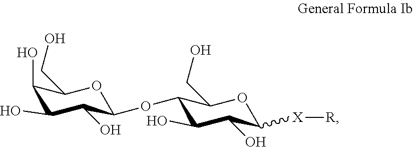

wherein
X and R are as defined for General Formula I in (1). [symbol]
   wherein glycosidic bond [symbol] is preferably a beta glycosidic bond.
(9) The method according to any one of (1) to (8), wherein the genetically modified cell has no β-galactosidase activity.
(10) The method according to any one of (1) to (6), wherein the glycosylated sphingoid base of interest is a compound of General Formula IIIa:

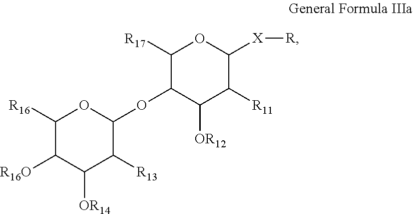

wherein
X and R are as defined for General Formula I in (1),
R₁₁ and R₁₃ are independently selected from the group consisting of OH, NH₂, NH-acyl and O-glycoside,
R₁₂, R₁₄ and R₁₅ are independently selected from the group consisting of hydrogen and a glycosyl moiety,
and
R₁₆ and R₁₇ are independently selected from the group consisting of CH₂—OH, CH₂O-glycoside and C₁₋₆ alkyl, preferably methyl.
(11) The method according to any one of (1) to (7), wherein the glycosylated sphingoid base of interest is a compound of General Formula IIIb:

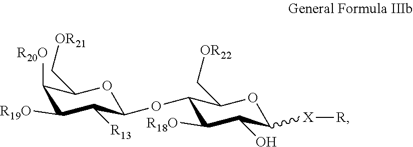

wherein
X and R are as defined for General Formula I in (1),
R₁₁ and R₁₃ are defined as for General Formula IIIa in (10),
and
R₁₈ to R₂₂ are independently selected from the group consisting of hydrogen or a glycosyl moiety.
(12) The method according to any one of (1) to (8), wherein the glycosylated sphingoid base of interest is a compound of General Formula IIIc:

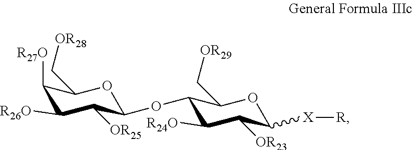

wherein
X and R are as defined for General Formula I in (1)
and
R₂₃ to R₂₉, are independently hydrogen or a glycosyl moiety.
(13) The method according to (8), wherein the glycosyltransferase enzyme is a α-2,3-sialyltransferase and the produced glycosylated sphingoid base of interest is compound of General Formula IV or a salt thereof:

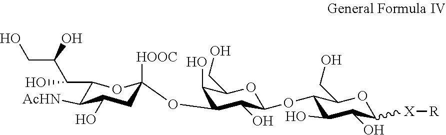

wherein
X and R are as defined for General Formula I in (1)
and
glycosidic bond [symbol] is preferably a beta glycosidic bond.
(14) The method according to (8), wherein the glycosyltransferase enzymes are α-2,8-sialyltransferase and α-2,3-sialyltransferase and the produced glycosylated sphingoid base of interest is compound of General Formula V or a salt thereof:

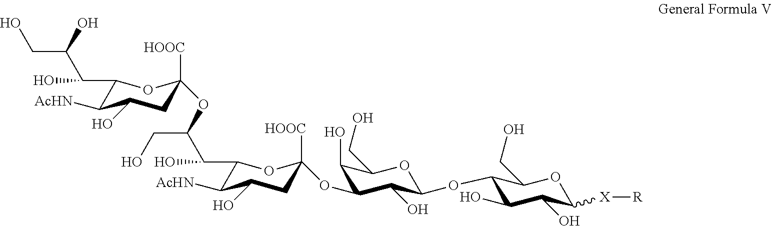

wherein
X and R are as defined for General Formula I in (1)
and
glycosidic bond [symbol] is preferably a beta glycosidic bond.
(15) The method according to (8), wherein the glycosyltransferase enzymes are β-1,4-N-acetylgalactosaminyltransferase, β-1,3-galactosyltransferase and α-2,3-sialyltransferase, and the produced glycosylated sphingoid base of interest is compound of General Formula VI or a salt thereof:

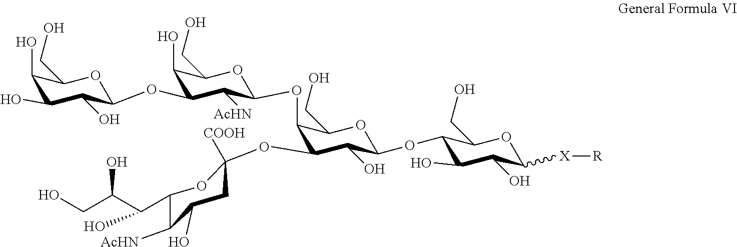

wherein
X and R are as defined for General Formula I in (1)
and
glycosidic bond [symbol] is preferably a beta glycosidic bond.
(16) The method according to any one of (1) to (15), wherein X is O and/or R is a group of formula 2a or of formula 2b

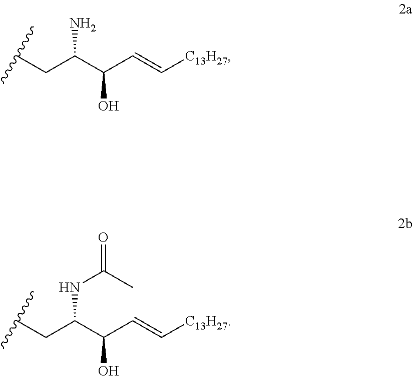

(17) Compound VII:

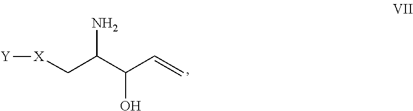

wherein X is O, linking Y via an O-glycosidic linkage,
and Y is selected from:
Galβ1-3GalNAcβ1-4(Neu5Acα2-3)Galβ1-4Glc1-,
Neu5Acα2-3Galβ1-3GalNAcβ1-4Galβ1-4Glc1-,
GalNAcβ1-4(Neu5Acα2-3)Galβ1-4Glc1-,
Neu5Acα2-3Galβ1-4Glc1-,
Neu5Acα2-3Galβ1-,
Neu5Acα2-3Galβ1-3GalNAcβ1-4(Neu5Acα2-3)Galβ1-4Glc1-,
Galβ1-3GalNAcβ1-4(Neu5Acα2-8Neu5Acα2-3)Galβ1-4Glc1-,
Neu5Acα2-8Neu5Acα2-3Galβ1-3GalNAcβ1-4Galβ1-4Glc1-,
Neu5Acα2-3Galβ1-3(Neu5Acα2-6)GalNAcβ1-4Galβ1-4Glc1-,
GalNAcβ1-4(Neu5Acα2-8Neu5Acα2-3)Galβ1-4Glc1-,
Neu5Acα2-8Neu5Acα2-3Galβ1-4Glc1-,
Neu5Acα2-8Neu5Acα2-3Galβ1-3GalNAcβ1-4(Neu5Acα2-3)Galβ1-4Glc1-,
Neu5Acα2-3Galβ1-3GalNAcβ1-4(Neu5Acα2-8Neu5Acα2-3)Galβ1-4Glc1-,
Galβ1-3GalNAcβ1-4(Neu5Acα2-8Neu5Acα2-8 Neu5Acα2-3)Galβ1-4Glc1-,
Neu5Acα2-3Galβ1-3(Neu5Acα2-6)GalNAcβ1-4(Neu5Acα2-3)Galβ1-4Glc1-,
GalNAcβ1-4(Neu5Acα2-8Neu5Acα2-8 Neu5Acα2-3)Galβ1-4Glc1-,
Neu5Acα2-8Neu5Acα2-8 Neu5Acα2-3Galβ1-4Glc1-,
Neu5Acα2-8Neu5Acα2-3Galβ1-3GalNAcβ1-4(Neu5Acα2-8Neu5Acα2-3)Galβ1-4Glc1-,
Neu5Acα2-3Galβ1-3(Neu5Acα2-6)GalNAcβ1-4(Neu5Acα2-8Neu5Acα2-3)Galβ1-4Glc1-,
Neu5Acα2-3Galβ1-3GalNAcβ1-4(Neu5Acα2-8Neu5Acα2-8Neu5Acα2-3) Galβ1-4Glc1-.
(18) Compound VIII:

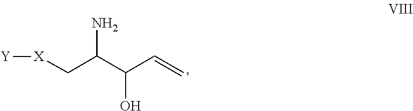

wherein X is O, linking Y via an O-glycosidic linkage,
and Y is selected from:
Galα1-4Galβ1-4Glc1-,
Galα1-3Galβ1-4Glc1-,
GalNAcβ1-3Galα1-4Galβ1-4Glc1-,
GalNAcβ1-3Galα1-3Galβ1-4Glc1-,
Galβ1-3GalNAcβ1-3Galα1-4Galβ1-4Glc1-,
Neu5Acα2-3Galβ1-3GalNAcβ1-3Galα1-4Galβ1-4Glc1-,
Neu5Acα2-3Galβ1-3(Neu5Acα2-6)GalNAcβ1-3Galα1-4Galβ1-4Glc1-,
GalNAcα1-3GalNAcβ1-3Galα1-4Galβ1-4Glc1-,
GalNAcβ1-3GalNAcβ1-3Galα1-4Galβ1-4Glc1-,
GalNAcα1-3GalNAcβ1-3(Galβ1-3GalNAcβ1-4)Galα1-4Galβ1-4Glc1-,
Galβ1-3GalNAcβ1-3Galα1-4Galβ1-4Glc1-,
Fucα1-2Galβ1-3GalNAcβ1-3Galα1-4Galβ1-4Glc1-,
GalNAcα1-3(Fucα1-2)Galβ1-3GalNAcβ1-3Galα1-4Galβ1-4Glc1-,
Galα1-3(Fucα1-2)Galβ1-3GalNAcβ1-3Galα1-4Galβ1-4Glc1-,
Galβ1-4(Fucα1-3)GlcNAcβ1-6(Galβ1-3)GalNAcβ1-3Galα1-4Galβ1-4Glc11-.
(19) Compound IX:

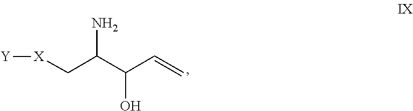

wherein X is O, linking Y via an O-glycosidic linkage,
and Y is selected from:
Galβ1-4GlcNAcβ1-3Galβ1-4Glc1-,
GlcNAcβ1-3Galβ1-4GlcNAcβ1-3Galβ1-4Glc1-,
Galβ1-4GlcNAcβ1-3Galβ1-4GlcNAcβ1-3Galβ1-4Glc1-,
Galβ1-4(Fucα1-3)GlcNAcβ1-3Galβ1-4Glc1-,
Galβ1-4(Fucα1-3)GlcNAcβ1-3Galβ1-4GlcNAcβ1-3Galβ1-4Glc1-,
Galβ1-4(Fucα1-3)GlcNAcβ1-3Galβ1-4(Fucα1-3)GlcNAcβ1-3Galβ1-4Glc1-,
Galβ1-4GlcNAcβ1-3(Galβ1-4GlcNAcβ1-6)Galβ1-4GlcNAcβ1-3Galβ1-4Glc1-,
Fucα1-2Galβ1-4GlcNAcβ1-3Galβ1-4Glc1-,
GalNAcβ1-3(Fucα1-2)Galβ1-4GlcNAcβ1-3Galβ1-4Glc1-,
GalNAcα1-3(Fucα1-2)Galβ1-4GlcNAcβ1-3Galβ1-4Glc1-,
Galα1-3(Fucα1-2)Galβ1-4GlcNAcβ1-3Galβ1-4Glc1-,
Fucα1-2Galβ1-3GalNAcα1-3(Fucα1-2)Galβ1-4GlcNAcβ1-3Galβ1-4Glc1-,
Fucα1-2Galβ1-4(Fucα1-3)GlcNAcβ1-3Galβ1-4Glc1-,
Fucα1-2Galβ1-4(Fucα1-3)GlcNAcβ1-3Galβ1-4GlcNAcβ1-3Galβ1-4Glc1-,
Galβ1-3(Fucα1-4)GlcNAcβ1-3Galβ1-4(Fucα1-3)GlcNAcβ1-3Galβ1-4Glc1-,
Galβ1-3Galβ1-4GlcNAcβ1-3Galβ1-4Glc1-,
GalNAcβ1-3Galβ1-4GlcNAcβ1-3Galβ1-4Glc1-,
Galα1-3Galβ1-4GlcNAcβ1-3(GalNAcβ1-4)Galβ1-4Glc11-,
GlcNAcβ1-3Galβ1-4(Fucα1-3)GlcNAcβ1-3Galβ1-4Glc1-,
Galα1-3Galβ1-4(Fucα1-3)GlcNAcβ1-3Galβ1-4Glc1-,
Galβ1-4Galβ1-4GlcNAcβ1-3Galβ1-4Glc1-,
GalNAcβ1-3Galα1-3Galβ1-4GlcNAcβ1-3Galβ1-4Glc11-.
(20) Compound X:

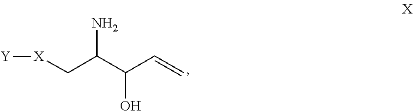

wherein X is O, linking Y via an O-glycosidic linkage,
and Y is selected from:
Galβ1-3GlcNAcβ1-3Galβ1-4Glc1-,
Galβ1-4GlcNAcβ1-3Galβ1-4Glc1-,
Galβ1-3GlcNAcβ1-3(Galβ1-4GlcNAcβ1-6)Galβ1-4Glc1-,
Galβ1-4GlcNAcβ1-3(Galβ1-4GlucNAcβ1-6)Galβ1-4Glc1-,
Fucα1-2Galβ1-4Glc1-,
Galβ1-4(Fucα1-3)Glc1-,
Fucα1-2Galβ1-4(Fucα1-3)Glc1-,
Fucα1-2Galβ1-3GlcNAcβ1-3Galβ1-4Glc1-,
Galβ1-3(Fucα1-4)GlcNAcβ1-3Galβ1-4Glc1-,
Galβ1-3(Fucα1-3)GlcNAcβ1-3Galβ1-4Glc1-,
Galβ1-3GlcNAcβ1-3Galβ1-4(Fucα1-3)Glc1-,
Fucα1-2Galβ1-3(Fucα1-4)GlcNacβ1-3Galβ1-4Glc1-,
Neu5Acα2-3Galβ1-4Glc1-,
Neu5Acα2-6Galβ1-4Glc1-,
Neu5Acα2-3Galβ1-4(Fucα1,3)Glc1-,
Neu5Acα2-3Galβ1-3GlcNAcβ1-3Galβ1-4Glc1-,
Galβ1-3(Neu5Acα2-6)GlcNAcβ1-3Galβ1-4Glc1-,
Neu5Acα2-6Galβ1-3GlcNAcβ1-3Galβ1-4Glc1-,
Neu5Acα2-3Galβ1-3(Neu5Acα2-6)GlcNAcβ1-3Galβ1-4Glc1-.
(21) Compound XI:

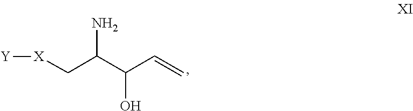

wherein X is O, linking Y via an O-glycosidic linkage,
and Y is selected from:
Galβ1-3Galβ1-4Glc1-,
Galβ1-4Galβ1-4Glc1-,
Galβ1-6Galβ1-4Glc1-,
Galβ1-3GalNAcβ1-4Galβ1-4Glc1-,
Fucα1-2Galβ1-3(Fucα1-4)GlcNAcβ1-3Galβ1-4Glc1-.

The present invention overcomes the drawbacks connected to the current techniques for the preparation of glycosylated sphingoid bases and provides a novel economically attractive method for the synthesis of a broad variety of glycosylated sphingoid bases in cells expressing the required enzymes. This method enables the production of glycosylated sphingoid bases having the desired stereo- and regiochemical configuration without the need for protecting group manipulations. Purification of the glycosylated sphingoid bases of interest can be achieved without the need for expensive and toxic reagents. Moreover, glycosyltransferases and glycosyl nucleotide donors are produced by the engineered cell and thus are readily available. These advantages enable an easy scale-up production of complex glycosylated sphingoid bases.

### DETAILED DESCRIPTION OF THE INVENTION

The glycosylated sphingoid bases of interest or analogues thereof of the present invention are produced starting from an exogenous precursor. The exogenous precursor is internalized by a cell that expresses one or more glycosyltransferases which catalyze the addition of further monosaccharide units to this exogenous precursor.

In the context of the present application, the following expressions are given a definition that should be taken into consideration with the claims and the description.

The term “substituted” means that the group in question is substituted with a group which typically modifies the general chemical characteristics of the group in question. Preferred substituents include but are not limited to halogen, nitro, amino, azido, oxo, hydroxyl, thiol, carboxy, carboxy ester, carboxamide, alkylamino, alkyldithio, alkylthio, alkoxy, acylamido, acyloxy, or acylthio, each of 1 to 6 carbon atoms, preferably of 1 to 3 carbon atoms. The substituents can be used to modify characteristics of the molecule as a whole such as molecule stability, molecule solubility and an ability of the molecule to form crystals. The person skilled in the art will be aware of other suitable substituents of a similar size and charge characteristics, which could be used as alternatives in a given situation.

In connection with the terms “alkyl” and “acyl”, the term “substituted” means that the group in question may be (is) substituted one or several times, preferably 1 to 3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo, C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any alkyl, alkoxy, and the like representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

The term “glycosyl moiety” when used herein is defined broadly to encompass a moiety derived from a monosaccharide unit or from an oligosaccharide (more than one monosaccharide unit), wherein the anomeric carbon of the monosaccharide or the anomeric carbon at the reducing end of the oligosaccharide is engaged in a glycosidic bond with another chemical entity. A glycosyl moiety having more than one monosaccharide may represent a linear or branched structure.

The monosaccharide unit can be any 5-9 carbon atom sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid).

The term “glycosyl moiety” for example includes the following moieties:

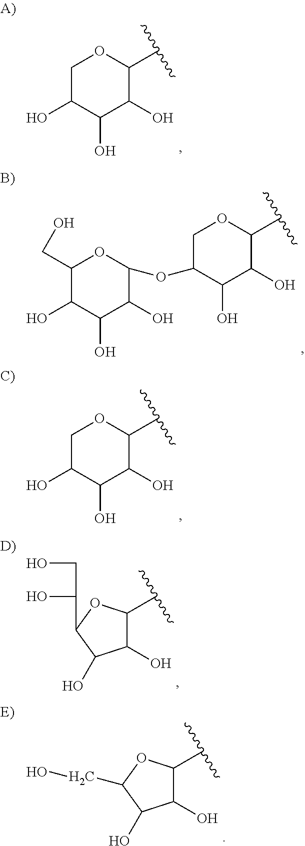 [SMILES: *C1OCC(O)C(O)C1O ; *C1OCC(OC2OC(CO)C(O)C(O)C2O)C(O)C1O ; *C1OCC(O)C(O)C1O ; *C1OC(C(O)CO)C(O)C1O ; *C1OC(CO)C(O)C1O]

The skilled person will understand that the term “O-glycosyl moiety” is a glycosyl moiety that is linked to another molecular moiety via an O-glycosidic linkage. Similarly, an “S-glycosyl moiety” is a glycosyl moiety that is linked to another molecular moiety via an S-glycosidic linkage, an “N-glycosyl moiety” is a glycosyl moiety that is linked to another molecular moiety via an N-glycosidic linkage and a “C-glycosyl moiety” is a glycosyl moiety that is linked to another molecular moiety via an C-glycosidic linkage.

The term “nucleic acid sequence” refers to a DNA fragment, which is either double-stranded or single stranded, or to a product of transcription of said DNA fragment, and/or to an RNA fragment. A nucleic acid sequence may be naturally present in a cell where it is expressed (termed as “endogenous nucleic acid sequence”) or may be introduced into a cell by recombinant nucleic acid techniques (termed as “heterologous nucleic acid sequence”). Commonly known recombinant nucleic acid techniques are e.g. described in Sambrook et al., Molecular Cloning: A Laboratory Manual, 2^{nd} Ed., Cold Spring Harbor Laboratory Press, Cold Spring Harbor, N.Y. (1989). A heterologous nucleic acid sequence may be a nucleic acid sequence that originates from a source foreign to the particular host cell, or, if from the same source, is modified from its original form. Thus, a heterologous nucleic acid sequence in a cell also includes a nucleic acid sequence that is endogenous to the particular cell but has been subjected to one or more modifications. Modification of a nucleic acid sequence may occur, e.g., by treating the DNA with a restriction enzyme to generate a DNA fragment that is capable of being operably linked to a promoter. Techniques such as site-directed mutagenesis are also useful for modifying a nucleic acid sequence.

The exogenous precursor or exogenous precursor molecule is a glycosylated sphingoid base or analogue thereof represented by General Formula I, preferably by General Formula Ia, more preferably by General Formula Ib, even more preferably by General Formula Ic as outlined in the present invention. The exogenous precursor molecule is modified by the method of the present invention in the way that one or more further monosaccharide units are attached to it by a glycosidic reaction. The glycosylated sphingoid base of interest or analogue thereof differs from the exogenous precursor in that the glycosylated sphingoid base of interest or analogue thereof comprises at least one more monosaccharide unit as compared to the exogenous precursor.

Glycosylated sphingoid bases are glycoconjugates comprising a glycosyl moiety covalently attached to a non-sugar moiety via an O-glycosidic linkage, wherein the non-sugar moiety comprises a sphingoid base backbone.

The term “sphingoid base” or “sphingoid base backbone” as used herein refers to N-, O- or C-substituted or unsubstituted 2-amino-1,3-dihydroxy-alkanes or N-, O- or C-substituted or unsubstituted 2-amino-1,3-dihydroxy-alkenes and are represented by “R” in General Formula I and by the sphingoid base backbone as shown in General Formulae IIa and in General Formula IIb, respectively:

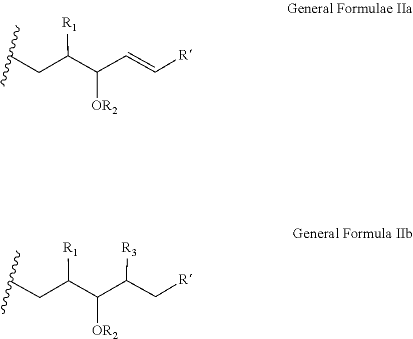

Accordingly, the term includes derivatives and analogues of naturally occurring sphingoid bases.

In the context of the present invention, an “analogue of a glycosylated sphingoid base” especially denotes a compound differing from a glycosylated sphingoid base at least in that the glycosyl moiety is attached to the non-sugar moiety via an N-, S-, or C-glycosidic linkage.

Sphingoid bases naturally present in humans are D-erythro-sphingosine (C₁₈H₃₇NO₂), 6-Hydroxy-D-erythro-sphingosine (C₁₈H₃₇NO₃), D-ribo-phytosphingosine (C₁₈H₃₉NO₃) or DL-erythro-Dihydrosphingosine (C₁₈H₃₉NO₂).

In one embodiment, R′ of General Formula IIa or IIb is an alkyl chain having 5-25 carbon atoms, more preferably 10-20 carbon atoms, even more preferably 13 carbon atoms; especially, R′ of General Formula IIa or IIb is C₁₃H₂₇ or CH(OH)C₁₂H₂₅, especially —C₁₃H₂₇ or —CH(OH)C₁₂H₂₅.

In another embodiment, R′ of General Formula IIa or IIb is an alkyl chain having 1-10 carbon atoms, more preferably 1 to 5 carbon atoms.

In a further embodiment, R′ of General Formula IIa or IIb is H.

R₁ of General Formula IIa or IIb is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure. Preferred cyclic structures are protecting groups, more preferably a phthaloyl protecting group, a tetrachlorophthaloyl protecting group or a vinylogous amide-type protecting group.

The glycosidic linkage of the exogenous precursor, of the derivative of the exogenous precursor or of the glycosylated sphingoid base of interest linking the glycosyl moiety to the non-sugar moiety represents either an alpha or a beta glycosidic bond. A beta glycosidic bond is preferred. The glycosidic bond linking the glycosyl moiety to the non-sugar moiety may be selected from an O-, S-, N- or C-glycosidic linkage, wherein X is in one embodiment O, S, NH or CH₂, respectively. Preferably, the glycosidic linkage is an O-glycosidic linkage and X is O. The glycosyl moiety (Y) is covalently attached to the non-sugar moiety by a glycosidic bond either directly to X, or optionally to a linker, which may be connected between the glycosyl moiety and the non-sugar moiety. Such a linker may be selected from an alkyl, an ether, an ester, an amine, an amide, a thioester, an oxygen, a carbon, a sulfur, a nitrogen and/or a thioether and may preferably have 1 to 4 atoms. Possible linkers include —NH—, —N(OH)—, —C(═O)—, —C(═S)—, —C(═NH)—, —C(═N—OH)—, —C(═O)—O—, —O—C(═O)—, —C(═O)—S—, —S—C(═O)—, —C(═S)—O—, —O—C(═S)—, —C(═S)—S—, —S—C(═S)—, —C(═O)—NH—, —NH—C(═O), —C(═NH)—O—, —O—C(═NH)—, —C(═S)—NH—, —NH—C(═S)—, —C(═NH)—S—, —S—C(═NH)—, —O—P(═O)—O—, —O—P(OH)(═O)—O—, oligoethylenglycol and/or oligopropylenglycol.

The term “glycosylated sphingoid base” as used herein potentially includes all derivatives of glycosylated sphingoid bases defined by R′, R₁, R₂ and R₃ according to General Formulae IIa or IIb.

The exogenous precursor can be synthesized chemically or enzymatically by any method of producing glycosidic linkages known to a skilled person. The exogenous precursor is preferably synthesized chemically. Example 1 provides an exemplary synthesis of exogenous precursor 1a as disclosed herein.

The cell used in the method according to the present invention may be prokaryotic or eukaryotic. It may e.g. be a bacterial cell, a yeast cell or a mammalian cell. Preferably, the cell used in the method according to the present invention is a microorganism, such as a bacterium or a yeast. More preferably, the bacterium is selected from the group comprising Escherichia coli, Bacillus spp. (e.g. Bacillus subtilis), Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, Lactobacillus spp., Lactococcus spp., Enterococcus spp., Bifidobacterium spp., Sporolactobacillus spp., Micromomospora spp., Micrococcus spp., Rhodococcus spp. and Pseudomonas, and the yeast is selected from the group comprising Saccharomyces cerevisiae, Schizosaccharomyces pombe, Pichia pastoris and Candida albicans. Most preferably, the genetically modified cell is an Escherichia coli (E. coli) cell.

The skilled person will understand that for the method of the present invention, the term “a genetically modified cell” does not intend to mean one single cell, but many cells, typically a cell clone showing the substantially the same genetic characteristics, that are cultured together in a culture medium. In the case of the cell originating from a mammal or from any other multicellular organism, the cells will be cultured in vitro isolated from the organism of origin. The expression “genetically modified” denotes that at least one alteration in the DNA sequence has been performed in the genome of the cell in order to give that cell a specific phenotype. The alteration in the DNA may e.g. be an introduction or a deletion of a DNA fragment in the genome. The alteration in the DNA sequence is herein especially achieved by the expression of a heterologous nucleic acid sequence, in particular a heterologous nucleic acid sequence encoding a glycosyltransferase enzyme. Genome editing may be performed e.g. by commonly known recombinant nucleic acid techniques as e.g. described in Sambrook et al., Molecular Cloning: A Laboratory Manual, 2^{nd} Ed., Cold Spring Harbor Laboratory Press, Cold Spring Harbor, N.Y. (1989). The CRISPR technology may also be used to perform genetic modifications.

The nucleic acid sequence encoding the glycosyltransferase enzyme may be an endogenous nucleic acid sequence or a heterologous nucleic acid sequence, preferably a heterologous nucleic acid sequence.

The genetically modified cell may comprise one or more than one nucleic acid sequence encoding one or more glycosyltransferase enzymes, such as two nucleic acid sequences encoding two or more glycosyltransferase enzymes, or three to five nucleic acid sequences encoding three to five glycosyltransferase enzymes. The nucleic acid sequences may be endogenous or heterologous. When more than one glycosyltransferase enzyme is expressed, the glycosyltransferase enzymes are preferably different ones and accordingly the encoding nucleic acid sequences are preferably different. When more than one glycosyltransferase is expressed and the encoding nucleic acid sequences are different, one or more encoding nucleic acid sequences may be heterologous and one or more encoding nucleic acid sequences may be endogenous.

In a preferred embodiment, the genetically modified cell further comprises one or more nucleic acid sequences encoding one or more epimerase enzymes, wherein the nucleic acid sequences may be endogenous or heterologous.

The origin of the heterologous nucleic acid sequences can be an animal (including humans), a plant, a yeast such as Saccharomyces cerevisiae, Saccharomyces pombe, Candida albicans, a bacterium such as E. coli, Bacillus subtilis, Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitidis, a protozoa such as trypanosoma, or a virus.

The nucleic acid sequences according to the present invention comprise or are a gene, a derivative of a gene or a transcription product of a gene, or a synthetic construct substantially identical to a gene. A derivative of a gene includes a nucleic acid sequence that is a fragment of a gene or a nucleic acid sequence that contains one or more mutations and/or deletions as compared to the original gene, or a cDNA; the mutations or deletions must not strongly impair the function of the encoded enzyme. A derivative of a gene is preferably at least 60% identical to a gene, more preferably at least 90% identical to a gene, even more preferably at least 95% identical to a wildtype gene. The value for gene identity is typically generated when compared and aligned for maximum correspondence, as measured using one of the following sequence comparison algorithms or by visual inspection. A synthetic construct substantially identical to a gene may be produced by synthesis techniques known to the skilled person.

Because of the degeneracy of the genetic code, a large number of functionally identical nucleic acids encode any given peptide or protein. For instance, the codons CGU, CGC, CGA, CGG, AGA and AGG all encode the amino acid arginine. Thus, at every position where an arginine is specified by a codon, the codon can be altered to any of the corresponding codons described without altering the encoded peptide or protein. A derivative of a gene or a synthetic construct substantially identical to a gene is a nucleic acid sequence is in one embodiment codon-optimized for expression in the genetically modified cell according to the present invention.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul el al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands.

In a preferred embodiment, one or more of the nucleic acid sequences according to the present invention are double-stranded DNA fragments. More preferably, the nucleic acid sequences according to the present invention are heterologous nucleic acid sequences. The heterologous nucleic acid sequence is may be placed in an expression cassette. The expression cassette comprises a promoter and the gene or a derivative thereof or synthetic construct to be transcribed. The promoter may be a constitutive or an inducible promoter. A preferred inducible promoter is the lac promoter. The promoter may be induced by addition of the inducer isopropyl β-D-thiogalactoside (IPTG) or by any other lactose analogue to the culture medium. Additional factors or effecting expression may also be used. Transcription start and termination signals, enhancers, and other DNA sequences that influence gene expression can also be included in an expression cassette. When more than one heterologous gene or derivative thereof or synthetic construct is expressed in the cell, the genes or derivatives thereof or synthetic constructs can be expressed on a single expression cassette or on multiple expression cassettes that are compatible and can be maintained in the same cell. When a single expression cassette is used for the expression of more than one heterologous gene or derivative thereof or synthetic construct, the heterologous genes or derivatives thereof or synthetic constructs may be placed under the same promoter, such as an operon, or under several promoters. When several promoters present in one or more expression cassettes are used for the expression of several heterologous genes or derivatives thereof of synthetic constructs, these promoters may be identical or different. Several different inducible promoters present in one or more expression cassettes may be induced by different inducers.

The expression cassette may in one embodiment be introduced into the cell by being placed on an expression vector. The expression vector typically further comprises a selection marker, including e.g. ampicillin or kanamycin.

A heterologous nucleic acid sequence can be expressed in the cell transiently or stably. For example, one expression vector can be used for one or several expression cassettes or more than one expression vector can be used for more than one expression cassette. Heterologous nucleic acid sequences according to the present invention can also be inserted into the chromosome of the cell, using methods known to those skilled in the art, including homologous recombination, site-specific recombination or transposon-mediated gene transposition. The CRISPR technology may also be used to insert one or more heterologous nucleic acid sequences or one or more expression cassettes into a specific locus of the chromosome of the cell. Combinations of expression cassettes in extrachromosomal vectors and expression cassettes inserted into a host cell chromosome can also be used.

Glycosyltransferases are enzymes that catalyze glycosylation reactions between a glycosyl donor, which is typically an activated sugar nucleotide (for Leloir glycosyltransferases), and a glycosyl acceptor, which is a nucleophilic biomolecule including a sugar, a protein or a lipid. Activated sugar nucleotides generally comprise a phosphorylated glycosyl residue attached to a nucleoside. The glycosyl residue of the donor is transferred to the acceptor by a glycosyltransferase, forming a glycosidic linkage.

The glycosyltransferase enzymes encoded by the nucleic acid sequences according to the method of the present invention are typically Leloir glycosyltransferases, capable of performing a glycosylation reaction between the exogenous precursor or a glycosylated derivative thereof, and an activated sugar nucleotide. The glycosyltransferase enzyme(s) according to the method of the present invention may be a glucosyltransferase, a galactosyltransferase, an N-acetylglucosaminyltransferase, an N-acetylgalactosaminyltransferase, a glucoronosyltransferase, a xylosyltransferase, a mannosyltransferase, a fucosyltransferase, a sialyltransferase, and the like, or combinations thereof. In a preferred embodiment, the one or more glycosyltransferase enzymes encoded by the one or more nucleic acid sequences according to the method of the present invention may be an α-2-0-fucosyltransferase, a β-1,3-N-acetylglucosaminyltransferase, a β-1,6-N-acetylglucosaminyltransferase, a β-1,3-galactosyltransferase, a β-1,4-galactosyltransferase, a β-1,4-N-acetylgalactosaminyltransferase, β-1,3-N-acetylgalactosaminyltransferase, a β-1,3-glucoronosyltransferase, an α-2,3-sialyltransferase, an α-2,6-sialyltransferase, an α-2,8-sialyltransferase, an α-1,2-fucosyltransferase, an α-1,3-fucosyltransferase, an α-1,4-fucosyltransferase, an α-1,4-galactosyltransferase, an α-1,3-galactosyltransferase or a combination thereof.

In some preferred embodiments, the one or more glycosyltransferase enzymes encoded by the one or more nucleic acid sequences according to the method of the present invention comprise one or more sialyltransferases (EC 2.4.99.-). In some more preferred embodiments, the one or more sialyltransferases comprise an α-2,3-sialyltransferase (β-galactoside α-2,3-sialyltransferase (EC 2.44.99.4)), an α-2,6-sialyltransferase (β-galactoside α-2,6-sialyltransferase (EC 2.44.99.1), an α-2,8-sialyltransferase (α-N-acetylneuraminate α-2,8-sialyltransferase (EC 2.44.99.8), or a combination thereof.

In some preferred embodiments, the one or more glycosyltransferase enzymes encoded by the one or more nucleic acid sequences according to the method of the present invention comprise one or more fucosyltransferases. In some more preferred embodiments, the one or more fucosyltransferases comprise an α-1,2-fucosyltransferase (type 1 galactoside α-1,2-fucosyltransferase (EC 2.4.1.69)), an α-1,3-fucosyltransferase (glycoprotein 3-α-L-fucosyltransferase (EC 2.4.1.214), an α-1,4-fucosyltransferase (EC 2.4.1.65), or a combination thereof.

In some preferred embodiments, the one or more glycosyltransferase enzymes encoded by the one or more nucleic acid sequences according to the method of the present invention comprise one or more fucosyltransferases and one or more sialyltransferases.

In a more preferred embodiment, the one or more glycosyltransferase enzymes encoded by the one or more nucleic acid sequences according to the method of the present invention is a β-1,4-N-acetylgalactosaminyltransferase, a β-1,3-galactosyltransferase, an α-2,3-sialyltransferase, an α-2,8-sialyltransferase or a combination thereof.

In an especially preferred first embodiment, the glycosyltransferase enzyme encoded by the nucleic acid sequence according to the method of the present invention is an α-2,3-sialyltransferase. The nucleic acid sequence according to the method of the present invention encoding the α-2,3-sialyltransferase may be the gene nst from Neisseria meningitidis (GenBank accession number U60660).

In an especially preferred second embodiment, the glycosyltransferase enzymes encoded by the nucleic acid sequences according to the method of the present invention are α-2,8-sialyltransferase and α-2,3-sialyltransferase. The nucleic acid sequence according to the method of the present invention encoding the α-2,8-sialyltransferase and α-2,3-sialyltransferase, respectively, may be the gene cstII from Campylobacter jejuni encoding the bifunctional α-2,3 and α-2,8 sialyltransferase (GenBank accession number AF400048).

In an especially preferred third embodiment, the glycosyltransferase enzymes encoded by the nucleic acid sequences according to the method of the present invention are β-1,4-N-acetylgalactosaminyltransferase, β-1,3-galactosyltransferase and α-2,3-sialyltransferase. The nucleic acid sequence according to the method of the present invention encoding the β-1,4-N-acetylgalactosaminyltransferase, β-1,3-galactosyltransferase and α-2,3-sialyltransferase, respectively, may be the gene cgtA from Campylobacter jejuni (AF130984), the gene from Campylobacter jejuni encoding the β-1,3-galactosyltransferase (AL111168), and gene nst from Neisseria meningitidis (GenBank accession number U60660) respectively.

The activated sugar nucleotide used for the glycosylation reaction of the present invention may e.g. be UDP-Glc, UDP-Gal, UDP-GlcNAc, UDP-GalNAc, UDP-glucuronic acid, UDP-Xyl, GDP-Man, GDP-Fuc and CMP-sialic acid. The activated sugar nucleotide used for the glycosylation reaction of the present invention is preferably selected from UDP-Gal, UDP-GalNAc and CMP-sialic acid. A skilled person knows that the choice of glycosyltransferase determines the sugar nucleotide possible as donor for the glycosylation reaction. When more than one different glycosyltransferase enzyme is expressed in the cell, also more than one different activated sugar nucleotide may be needed to be present in the cell, depending on whether the different glycosyltransferases use the same or different sugar nucleotides as donors.

The activated sugar nucleotide is typically synthesized by a suitable nucleotidylyltransferase from a carbon substrate. Accordingly, the genetically modified cell used for the method of the present invention preferably comprises a nucleic acid sequence encoding a nucleotidylyltransferase capable of producing the desired activated sugar nucleotide. The nucleic acid sequence may be naturally present in the cell or may be heterologously expressed after introduction into the cell by means of recombinant techniques generally known to the skilled person. Preferred nucleotidylyltransferases include uridylyltransferases, guanylyltransferases and cytitidylyltransferases. The carbon substrate may be used from an exogenous addition to the genetically modified cell and/or may originate from a salvage pathway. Preferred carbon substrates include glycerol, glucose, glycogen, fructose, maltose, starch, cellulose, pectin, sucrose or chitin.

When a α-2,3-sialyltransferase and/or α-2,8 sialyltransferase are expressed in the genetically modified cell according to the present invention, CMP-sialic acid is typically used as donor for the glycosylation reactions on the exogenous precursor and on a glycosylated derivative thereof, respectively. CMP sialic acid may be produced in the cell from UDP-GlcNAc by the expression of genes encoding a CMP-Neu5Ac synthetase, a sialic acid synthase and a GlcNAc-6-phosphate 2 epimerase while eliminating the activity of N-acetylmannosamine (ManNAc) kinase and N-acetyl-D-neuraminic acid (Neu5Ac) aldolase. The CMP-Neu5Ac synthetase is preferably encoded by the gene neuA from Campylobacter jejuni (AF400048), the sialic acid synthase is preferably encoded by the gene neuB from Campylobacter jejuni (AF400048) and the GlcNAc-6-phosphate 2 epimerase is preferably encoded by the gene neuC from Campylobacter jejuni (AF400048). The genes may be heterologously expressed in the genetically modified cell of the present invention, while, where e.g. E. coli is the genetically modified cell, the nanKETA genes have been inactivated.

When a β-1,4-N-acetylgalactosaminyltransferase, a β-1,3-galactosyltransferase and a α-2,3-sialyltransferase are expressed in the genetically modified cell, UDP-GalNAc is typically used as donor for the glycosylation reaction performed by β-1,4-N-acetylgalactosaminyltransferase, UDP-galactose is typically used as donor for the glycosylation reaction performed by β-1,3-galactosyltransferase, and CMP-sialic acid is typically used as donor for the glycosylation reaction performed by α-2,3-sialyltransferase.

UDP-GalNAc may be produced in the genetically modified cell by the expression of a gene encoding a UDP-GlcNAc-4-epimerase, such as the wbpP gene from Pseudomonas aeruginosa (AF035937) or the gne gene from Campylobacter jejuni (AL111168). CMP sialic acid may be produced in the genetically modified cell from UDP-GlcNAc by the expression of genes encoding a CMP-Neu5Ac synthetase, a sialic acid synthase and a GlcNAc-6-phosphate 2 epimerase while eliminating the activity of N-acetylmannosamine (ManNAc) kinase and N-acetyl-D-neuraminic acid (Neu5Ac) aldolase. The CMP-Neu5Ac synthetase is preferably encoded by the gene neuA from Campylobacter jejuni (AF400048), the sialic acid synthase is preferably encoded by the gene neuB from Campylobacter jejuni (AF400048) and the GlcNAc-6-phosphate 2 epimerase is preferably encoded by the gene neuC from Campylobacter jejuni (AF400048). The genes may be heterologously expressed in the genetically modified cell of the present invention, while, where e.g. E. coli is the genetically modified cell, the nanKETA genes have been inactivated.

In step b) of the method of the present invention, the genetically modified cell is cultured in a culture medium. When the cell of the present invention is a bacterial or a yeast cell, the culturing corresponds to a fermentation process and the “culture medium” may be also termed as “fermentation broth”.

A fermentation process typically includes two phases:
1. a first phase of exponential cell growth ensured by a carbon-based substrate, and
2. a second phase of cell growth limited by a carbon-based substrate which is added continuously.

The fermentation process may preferably further comprise a third phase (3.) of slowed cell growth obtained by continuously adding to the culture an amount of carbon-based substrate that is less than the amount of carbon-based substrate added in the second phase of the fermentation process so as to further increase the produced compound. More preferably, the amount of carbon-based substrate added during the third phase of the fermentation is at least 30% less than the amount of the carbon-based substrate added during the second phase of the fermentation.

During fermentation, the exogenous precursor may be added to the culture medium at one time point, stepwise or continuously. The pure precursor in solid or in liquid form or a concentrated aqueous solution of the precursor can be added at one time point at the start of fermentation or at the end of the first phase of exponential growth.

The carbon-based substrate may be selected from sucrose, glycerol and glucose. The carbon-based substrate added during the second phase is preferably glycerol.

The culturing is preferably performed under conditions allowing the production of a culture with a high cell density. The skilled person is aware of such conditions, including e.g. pH control and pO₂ control. pO₂ is preferably more than 10%, more preferably more than 20%, even more preferably more than 40% with air flow and stirring.

The first phase of the fermentation process may be performed at a reaction temperature of e.g. 30° C., 31° C., 32° C., 33° C., 34° C., 35° C. or 36° C.

The second phase of the fermentation process may be performed at a reaction temperature of e.g. 25° C., 26° C., 27° C., 28° C., 29° C. or 30° C.

The pH regulated may be kept stable by the addition of e.g. aqueous NH₄₀H, NaOH or KOH solution.

In step b) i. of the method of the present invention, the exogenous precursor is internalized by the cell. The internalization step must not affect the basic and vital functions or destroy the integrity of the cell. The exogenous precursor molecule may be internalized solely or also via a passive transport during which the exogenous precursor molecule diffuses passively across the plasma membrane of the cell. The flow is directed by the concentration difference in the extra- and intracellular space with respect to the exogenous precursor molecule to be internalized, which exogenous precursor molecule is supposed to pass from the place of higher concentration to the zone of lower concentration tending towards an equilibrium. Typically, the genetically modified cell comprises a transporter protein, which internalizes the exogenous precursor molecule via active transport. Different transporter proteins have specificities for different sugar moieties of the molecules to be internalized. This specificity may be altered by mutation by means of common recombinant DNA techniques. Preferably, the internalization of the exogenous precursor molecule is performed via a transporter protein.

The internalized precursor is then subjected to a glycosylation reaction according to step b) ii) of the method of the present invention. For the glycosylation reaction of the present invention taking place in the cell, the exogenous precursor molecule serves as glycosyl acceptor. The addition of one monosaccharide unit to the exogenous precursor molecule is performed by a glycosyltransferase. The resulting molecule is termed in the present context as “glycosylated derivative of the exogenous precursor” or as “glycosylated sphingoid base of interest or analogue thereof”, depending on whether the molecule is subjected to at least one further glycosylation reaction in the cell (then termed as “glycosylated derivative of the exogenous precursor” or just “glycosylation derivative”) or whether it is the final molecule to be produced and subjected to step c) of the method of the present invention (then termed as “glycosylated sphingoid base of interest or analogue thereof”). If more than one glycosylation reaction is performed in the cell, the “glycosylation derivative” is the acceptor molecule for the second and every further glycosylation reaction. One to five glycosylation reactions are preferably performed in the cell. The monosaccharide unit that are added during a second and any further glycosylation reaction may be identical or different. The skilled person will understand that the addition of different monosaccharide unit is performed by different glycosyltransferases, which are encoded by different nucleic acid sequences, using different activated sugar nucleotides as donor molecule. The addition of identical monosaccharide units is typically also performed by different glycosyltransferases, which are encoded by different nucleic acid sequences, using however the same activated sugar nucleotides as donor molecule. Accordingly, when the glycosylated sphingoid base of interest or analogue thereof comprises at least two more monosaccharide units as compared to the exogenous precursor, those monosaccharide units are either identical or different from each other.

The exogenous precursor is a compound of General Formula I

Y—X—R General Formula I,

wherein
Y is a glycosyl moiety,
X is O, S, NH or CH₂, linking Y to R by an O-, S-, N- or C-glycosidic linkage, respectively, wherein the glycosidic linkage is preferably a beta-glycosidic linkage,
and R is a group of General Formula IIa or General Formula IIb:

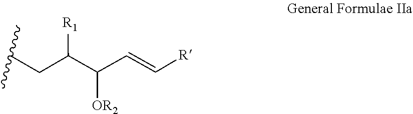

wherein
R′ is H, aryl or an alkyl chain having 1-43 carbon atoms, which may be a straight chain or branched, and/or which may be saturated or contain one or more double and/or triple bonds, and/or which may contain one or more functional groups, the functional group being preferably selected from the group consisting of a hydroxyl group, an alkoxy group, an acyloxy group, a primary, secondary or tertiary amine, an acylamido group, a thiol group, a thioether or a phosphorus-containing functional group,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl;

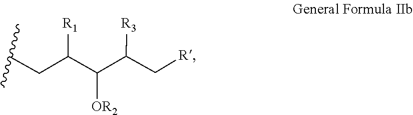

wherein R′, R₁, R₂, are as defined in General Formula IIa, and
R₃ is H, OH or OR₆, wherein R₆ is selected from substituted or unsubstituted alkyl or substituted or unsubstituted acyl;
Glycosyl moiety Y of General Formula I is in a preferred embodiment a monosaccharide moiety, a disaccharide moiety or a trisaccharide moiety, more preferably a monosaccharide moiety or a disaccharide moiety. Glycosyl moiety Y of General Formula I is in a most preferred embodiment a disaccharide moiety.

The glycosyl moiety of the exogenous precursor of the present invention is preferably lactose (Lac, Galβ1-4Glc-) and the exogenous precursor according to the present invention is preferably a compound of General Formula Ic, wherein the glycosidic bond [symbol] is preferably a beta glycosidic bond, more preferably the following compound of General Formula Id or Ie, even more preferably the following compound 1a or 1b:

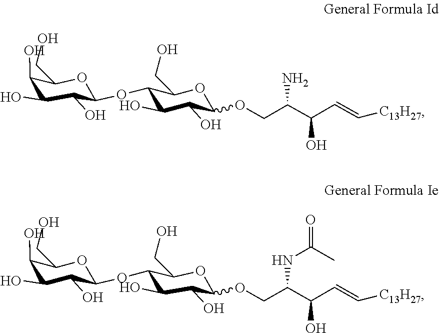

wherein the glycosidic bond [symbol] of Id and Ie is preferably a beta glycosidic bond;

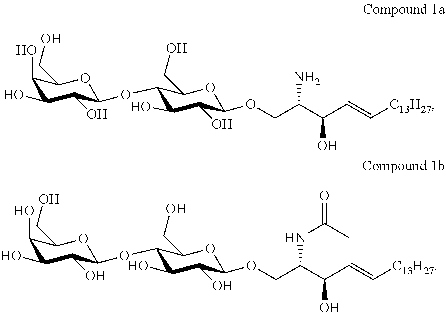

The glycosyl moiety of the glycosylated sphingoid base of interest or analogue thereof of the present invention is preferably selected from the following compounds or from salts thereof:

Galβ1-3GalNAcβ1-4(Neu5Acα2-3)Galβ1-4Glc1-
Neu5Acα2-3Galβ1-3GalNAcβ1-4Galβ1-4Glc1-
GalNAcβ1-4(Neu5Acα2-3)Galβ1-4Glc1-
Neu5Acα2-3Galβ1-4Glc1-
Neu5Acα2-3Galβ1-4Glc1-
Neu5Acα2-3Galβ1-
Neu5Acα2-3Galβ1-3GalNAcβ1-4(Neu5Acα2-3)Galβ1-4Glc1-
Galβ1-3GalNAcβ1-4(Neu5Acα2-8Neu5Acα2-3)Galβ1-4Glc1-
Neu5Acα2-8Neu5Acα2-3Galβ1-3GalNAcβ1-4Galβ1-4Glc1-
Neu5Acα2-3Galβ1-3(Neu5Acα2-6)GalNAcβ1-4Galβ1-4Glc1-
GalNAcβ1-4(Neu5Acα2-8Neu5Acα2-3)Galβ1-4Glc1-
Neu5Acα2-8Neu5Acα2-3Galβ1-4Glc1-
Neu5Acα2-8Neu5Acα2-3Galβ1-3GalNAcβ1-4(Neu5Acα2-3)Galβ1-
4Glc1-
Neu5Acα2-3Galβ1-3GalNAcβ1-4(Neu5Acα2-8Neu5Acα2-3)Galβ1-
4Glc1-
Galα1-4Galβ1-4Glc1-
Galα1-3Galβ1-4Glc1-
GalNAcβ1-3Galα1-4Galβ1-4Glc1-
GalNAcβ1-3Galα1-3Galβl-4Glcl-
Galβ1-3GalNAcβ1-3Galα1-4Galβ1-4Glc1-
Neu5Acα2-3Galβ1-3GalNAcβ1-3Galα1-4Galβ1-4Glc1-
Neu5Acα2-3Galβ1-3(Neu5Acα2-6)GalNAcβ1-3Galα1-4Galβ1-4Glc1-
GalNAcα1-3GalNAcβ1-3Galα1-4Galβ1-4Glc1-
GalNAcβ1-3GalNAcβ1-3Galα1-4Galβ1-4Glc1-
GalNAcα1-3GalNAcβ1-3(Galβ1-3GalNAcβ1-4)Galα1-4Galβ1-4G1c1-
Galβ1-3GalNAcβ1-3Galα1-4Galβ1-4Glc1-
Fucα1-2Galβ1-3GalNAcβ1-3Galα1-4Galβ1-4Glc1-
GalNAcα1-3(Fucα1-2)Galβ1-3GalNAcβ1-3Galα1-4Galβ1-4G1c1-
Galα1-3(Fucα1-2)Galβ1-3GalNAcβ1-3Galα1-4Galβ1-4Glc1-
Galβ1-4(Fucα1-3)GlcNAcβ1-6(Galβ1-3)GalNAcβ1-3Galα1-4Galβ1-
4Glc1-
Galβ1-4GlcNAcβ1-3Galβ1-4Glc1-
GlcNAcβ1-3Galβ1-4GlcNAcβ1-3Galβ1-4Glc1-
Galβ1-4GlcNAcβ1-3Galβ1-4GlcNAcβ1-3Galβ1-4Glc1-
Galβ1-4(Fucα1-3)GlcNAcβ1-3Galβ1-4Glc1-
Galβ1-4(Fucα1-3)GlcNAcβ1-3Galβ1-4GlcNAcβ1-3Galβ1-4Glc1-
Galβ1-4(Fucα1-3)GlcNAcβ1-3Galβ1-4(Fucα1-3)GlcNAcβ1-3Galβ1-
4Glc1-
Galβ1-4GlcNAcβ1-3(Galβ1-4GlcNAcβ1-6)Galβ1-4GlcNAcβ1-3Galβ1-
4Glc1-
Fucα1-2Galβ1-4G1cNAcβ1-3Galβ1-4Glc1-
GalNAcβ1-3(Fucα1-2)Galβ1-4GlcNAcβ1-3Galβ1-4Glc1-
GalNAcα1-3(Fucα1-2)Galβ1-4GlcNAcβ1-3Galβ1-4Glc1
Galα1-3(Fucα1-2)Galβ1-4GlcNAcβ1-3Galβ1-4Glc1-
Fucα1-2Galβ1-3GalNAcα1-3(Fucα1-2)Galβ1-4GlcNAcβ1-3Galβ1-
4Glc1-
Fucα1-2Galβ1-4(Fucα1-3)GlcNAcβ1-3Galβ1-4Glc1-
Fucα1-2Galβ1-4(Fucα1-3)GlcNAcβ1-3Galβ1-4GlcNAcβ1-3Galβ1-
4Glc1-
Galβ1-3(Fucα1-4)GlcNAcβ1-3Galβ1-4(Fucα1-3)GlcNAcβ1-3Galβ1-
4Glc1-
Galβ1-3Galβ1-4GlcNAcβ1-3Galβ1-4Glc1-
GalNAcβ1-3Galβ1-4GlcNAcβ1-3Galβ1-4Glc1-
Galα1-3Galβ1-4GlcNAcβ1-3(GalNAcβ1-4)Galβ1-4Glc1-
GlcNAcβ1-3Galβ1-4(Fucα1-3)GlcNAcβ1-3Galβ1-4Glc1-
Galα1-3Galβ1-4(Fucα1-3)GlcNAcβ1-3Galβ1-4Glc1-
Galβ1-4Galβ1-4GlcNAcβ1-3Galβ1-4Glc1-
GalNAcβ1-3Galα1-3Galβ1-4GlcNAcβ1-3Galβ1-4Glc1-
Galα1-3Galβ1-4GlcNAcβ1-3Galβ1-4Glc1-
Fucα1-2Galβ1-3(Fucα1-4)GlcNAcβ1-3Galβ1-4Glc1-
Fucα1-2Galβ1-4Glc1-
Galβ1-3Galβ1-4Glc1-
Galβ1-4Galβ1-4Glc1-
Galβ1-6Galβ1-4Glc1-
Galβ1-3GalNAcβ1-4Galβ1-4Glc1-
Galβ1-3GlcNAcβ1-3Galβ1-4Glc1-
Galβ1-4GlcNAcβ1-3Galβ1-4Glc1-
Galβ1-3GlcNAcβ1-3(Galβ1-4GlcNAcβ1-6)Galβ1-4Glc1-
Galβ1-4GlcNAcβ1-3Galβ1-4GlucNAcβ1-3Galβ1-4Glc1-
Galβ1-4(Fucα1-3)Glc1-
Fucα1-2Galβ1-4(Fucα1-3)Glc1-
Fucα1-2Galβ1-3GlcNAcβ1-3Galβ1-4Glc1-
Galβ1-3(Fucα1-4)GlcNAcβ1-3Galβ1-4Glc1-
Galβ1-3(Fucα1-3)GlcNAcβ1-3Galβ1-4Glc1-
Galβ1-3GlcNAcβ1-3Galβ1-4(Fucα1-3)Glc1-
Neu5Acα2-3Galβ1-4Glc1-
Neu5Acα2-6Galβ1-4Glc1-
Neu5Acα2-3Galβ1-4(Fucα1,3)Glc1-
Neu5Acα2-3Galβ1-3GlcNAcβ1-3Galβ1-4Glc1-
Galβ1-3(Neu5Acα2-6)GlcNAcβ1-3Galβ1-4Glc1-
Neu5Acα2-6Galβ1-3GlcNAcβ1-3Galβ1-4Glc1-
Neu5Acα2-3Galβ1-3(Neu5Acα2-6)GlcNAcβ1-3Galβ1-4Glc1-

In some embodiments, the glycosyl moiety of the glycosylated sphingoid base of interest or analogue thereof corresponds to the glycosyl moiety of a ganglioside, such as GM1a, GM1b, GM2, GM3, GD3, GM4, GD1a, GD1b.

In some embodiments, the glycosyl moiety of the glycosylated sphingoid base of interest or analogue thereof corresponds to the glycosyl moiety of a human milk oligosaccharide (HMNO), such as LNT, LNnT, LNH, LNnH, 2′FL, 3′FL, DFL, LNFP-J, LNFP-JJ, 3′ SL, 6′ SL.

The glycosyl moiety of the glycosylated sphingoid base of interest or analogue thereof of the present invention is more preferably selected from Neu5Acα2-3Galβ1-4Glc1-, Neu5Acα2-8Neu5Acα2-3Galβ1-4Glc1- and Galβ1-3GalNAcβ1-4(Neu5Acα2-3)Galβ1-4Glc1-, whose glycosyl moieties correspond to the glycosyl moieties of GM3, GD3 and GM1a, respectively. The glycosylated sphingoid base of interest or analogue thereof is preferably selected from

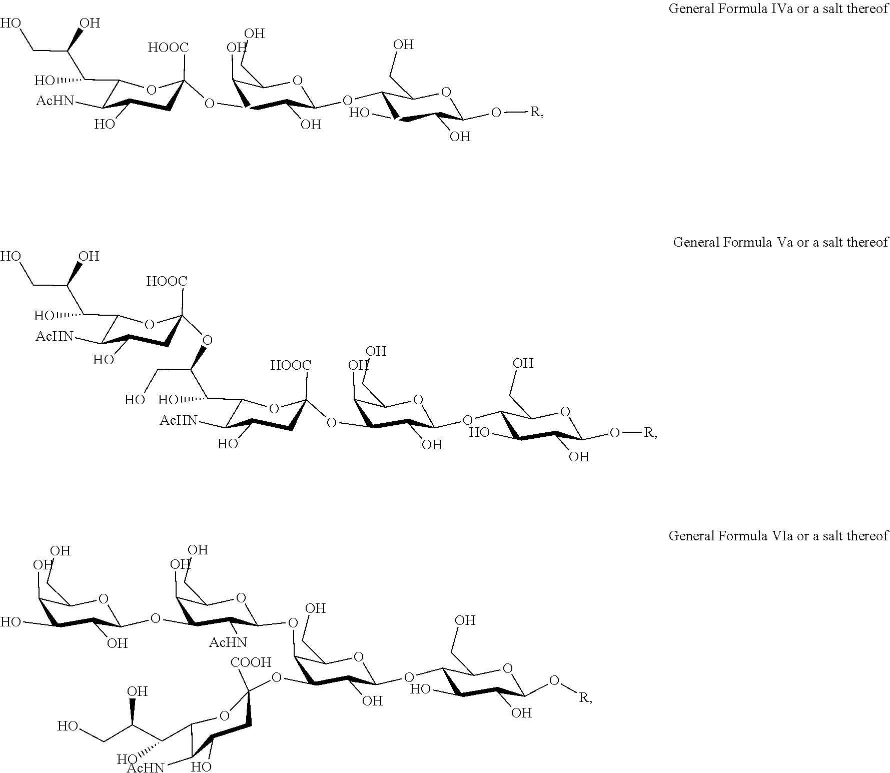

wherein
R of General Formula IVa, Va and VIa is a group of General Formula IIa or General Formula IIb:

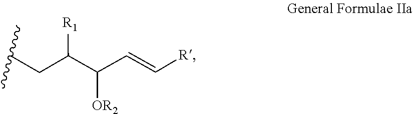

wherein
R′ is H, aryl or an alkyl chain having 1-43 carbon atoms, which may be a straight chain or branched, and/or which may be saturated or contain one or more double and/or triple bonds, and/or which may contain one or more functional groups, the functional group being preferably selected from the group consisting of a hydroxyl group, an alkoxy group, an acyloxy group, a primary, secondary or tertiary amine, an acylamido group, a thiol group, a thioether or a phosphorus-containing functional group,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl;

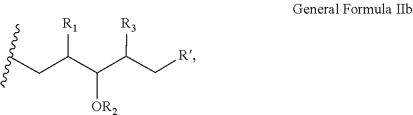

wherein R′, R₁, R₂, are as defined in General Formula IIa, and
R₃ is H, OH or OR₆, wherein R₆ is selected from substituted or unsubstituted alkyl or substituted or unsubstituted acyl.

The glycosylated sphingoid base of interest or analogue thereof is more preferably selected from:

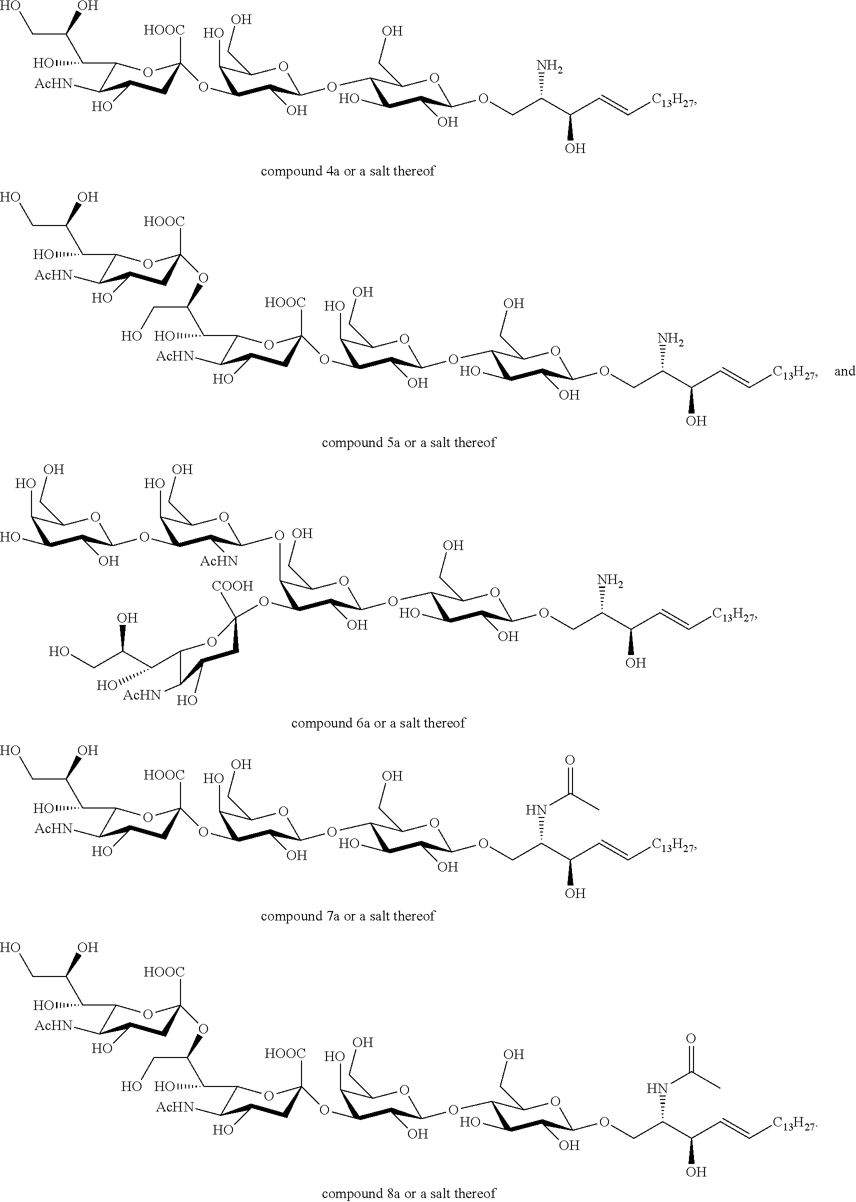

The skilled person will understand that with the exception of the glycosyl moiety, the preferred embodiments as mentioned for the exogenous precursor equally relate to the glycosylated sphingoid base of interest or analogue thereof, the latter ones being produced from the former ones.

R′ of the exogenous precursor of General Formula IIa and IIb and of the glycosylated sphingoid base of interest or analogue thereof is in a preferred embodiment an alkyl chain having 1-20 carbon atoms, especially an alkyl chain having 13 carbon atoms.

The exogenous precursor and the glycosylated sphingoid base of interest is preferably a group of formula XII, XIII, IX or XV:

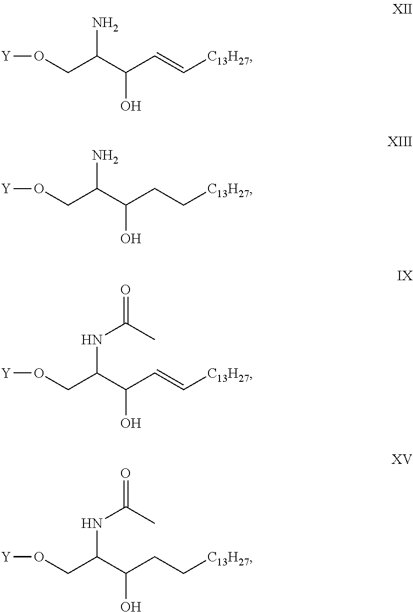

wherein Y of XII, XIII, IX and XI is a glycosyl moiety.

In a preferred embodiment, the genetically modified cell lacks any enzymatic activity which would degrade the exogenous precursor, the glycosylated derivatives of the exogenous precursor and/or the glycosylated sphingoid base of interest or analogue thereof. In a more preferred embodiment, the endogenous gene encoding for β-galactosidase (EC 3.2.1.23) and/or the endogenous gene encoding for N-acetylmannosamine kinase (EC 2.7.1.60) is/are inactivated in the genetically modified cell, so as no functional enzyme can be produced. Most preferably, the endogenous gene encoding for β-galactosidase is inactivated in the genetically modified cell. Accordingly, where the genetically modified cell is E. coli, the gene lacZ is preferably inactivated. The gene encoding for α-galactosidase (in E. coli the gene melA) may also be inactivated.

Step c) of the method of the present invention relates to the isolation of the glycosylated sphingoid base of interest or the analogue thereof from the cell and/or from the culture medium. Step c) may be an optional step of the method of the present invention. The glycosylated sphingoid base of interest or the analogue thereof of the method of the present invention can accumulate both in the intra- and extracellular matrix. Glycosylated sphingoid bases having more monosaccharide units tend to accumulate in the cell, while glycosylated sphingoid bases having less monosaccharide units are rather exported from the cell. When exported, the glycosylated sphingoid base of interest or analogue thereof may be exported from the cell via passive transport, by diffusing outside across the cell membrane into the culture medium. The export may be facilitated or mediated by sugar efflux transporters. A sugar efflux transporter may be naturally present in the cell or may be provided in form of a heterologous nucleic acid sequence encoding for the sugar efflux transporter produced by recombinant techniques known to the skilled person. The endogenous or heterologous nucleic acid sequence encoding for the sugar efflux transporter may in one embodiment be mutated by means of known recombinant techniques or may be overexpressed to increase the specificity towards the glycosyl moiety of the glycosylated sphingoid base of interest or analogue thereof to be secreted.

For the isolation step, the culture medium is preferably separated from the cells by filtration or centrifugation. When the glycosylated sphingoid base of interest or analogue thereof is mainly exported from the cell, it is mainly present in the supernatant containing the culture medium and purified and isolated therefrom by means of standard separation, purification and isolation techniques such as crystallization, precipitation and chromatography (e.g. silica gel chromatography, reverse phase chromatography, size exclusion chromatography, gel and/or ion exchange resin, etc.). When the glycosylated sphingoid base of interest or analogue thereof accumulates mainly inside the cell, the separated cells are preferably permeabilized. For that, the cells are resuspended in water and subjected to heat and/or acid or base treatment. Sodium hydroxide may be used for a base treatment and sulfuric acid may be used for acid treatment. The glycosylated sphingoid base of interest or analogue thereof is then separated from the treated cells by filtration and purified and isolated from the supernatant by means of standard separation, purification and isolation techniques such as gel and/or ion exchange resin chromatography. The supernatant containing the product from the culture medium may in one embodiment be combined with the supernatant containing the product from the lysed cells. Also, in this embodiment, the product may be purified and isolated from the combined supernatant by means of standard separation, purification and isolation techniques such as gel and or/ion exchange resin chromatography.

The invention relates in one preferred embodiment to a method for producing a compound of General Formula IV, preferably lyso-GM3, (4a), the method comprising the steps of:
a) Providing an exogenous precursor and a genetically modified cell, wherein the exogenous precursor is a compound of General Formula Id, preferably compound 1a, and wherein the genetically modified cell is an E. coli lacZ⁻ cell comprising a heterologous nucleic acid sequence encoding an α-2,3-sialyltransferase and one or more heterologous nucleic acid sequences encoding a CMP-Neu5Ac synthetase, a sialic acid synthase and a GlcNAc-6-phosphate 2 epimerase, and wherein the nanKETA genes of the E. coli lacZ⁻ cell have been inactivated,
b) Culturing said genetically modified cell in a culture medium comprising said exogenous precursor, whereby
   i. the exogenous precursor is internalized by the cell, and
   ii. one glycosylation reaction is performed on the internalized exogenous precursor, to form a compound of General Formula IV, preferably compound 4a,
c) Optionally isolating the compound of General Formula IV, preferably compound 4a, from the genetically modified cell and/or the culture medium.

The invention relates in another preferred embodiment to a method for producing a compound of General Formula V, preferably lyso-GD3, (5a), the method comprising the steps of:
a) Providing an exogenous precursor and a genetically modified cell, wherein the exogenous precursor is a compound of General Formula Id, preferably compound 1a, and wherein the genetically modified cell is an E. coli lacZ⁻ cell comprising one or more heterologous nucleic acid sequences encoding an α-2,3-sialyltransferase and an α-2,8-sialyltransferase and one or more heterologous nucleic acid sequences encoding a CMP-Neu5Ac synthetase, a sialic acid synthase and a GlcNAc-6-phosphate 2 epimerase, and wherein the nanKETA genes of the E. coli lacZ⁻ cell have been inactivated,
b) Culturing said genetically modified cell in a culture medium comprising said exogenous precursor, whereby
   i. the exogenous precursor is internalized by the cell, and
   ii. two glycosylation reactions are performed on the internalized exogenous precursor, to form a compound of General Formula V, preferably compound 5a,
c) Optionally isolating the compound of General Formula V, preferably compound 5a, from the genetically modified cell and/or the culture medium.

In one embodiment of the method of the present invention, the exogenous precursor is a compound of General Formula Ic:

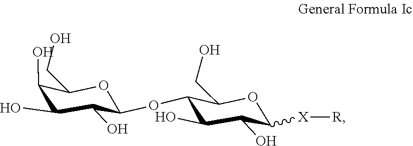

the genetically modified cell is an E. coli lacZ⁻ cell, wherein the nanKETA genes have been inactivated, the encoded glycosyltransferase enzymes are an α-2,3-sialyltransferase, an α-2,8-sialyltransferase, a β-1,4-GalNAc transferase and a β-1,3-galactosyltransferase. The nucleic acid sequences encoding said glycosyltransferase enzymes may be cstII (encoding the bifunctional α-2,3 α-2,8-sialyltransferase from C. jejuni), cgtAII from C. jejuni and cgtB from C. jejuni.

In another embodiment of the method of the present invention, the exogenous precursor is a compound of General Formula Ic:

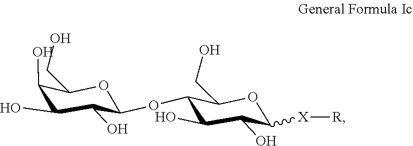

the genetically modified cell is an E. coli lacZ⁻ cell, wherein the nanKETA genes have been inactivated, the encoded glycosyltransferase enzymes are an α-2,3-sialyltransferase, an α-2,8-sialyltransferase, a β-1,4-GalNAc transferase, a β-1,3-galactosyltransferase, and a UDP-GlcNAc-4-epimerase is expressed. The nucleic acid sequences encoding said glycosyltransferase enzymes may be cstII (encoding the bifunctional α-2,3 α-2,8-sialyltransferase from C. jejuni), cgtAII from C. jejuni and cgtB from C. jejuni, and the nucleic acid sequence encoding the UDP-GlcNAc-4-epimerase may be gne from C. jejuni or wbpP from P. aeruginosa.

In a further embodiment of the method of the present invention, the exogenous precursor is a compound of General Formula Id:

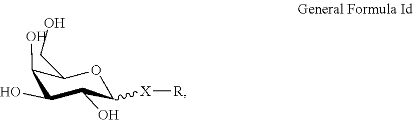

the genetically modified cell is an E. coli lacZ⁻ cell, wherein the nanKETA genes have been inactivated, the encoded glycosyltransferase enzymes are an α-2,3-sialyltransferase, an α-2,8-sialyltransferase, a β-1,4-GalNAc transferase, a β-1,3-galactosyltransferase, and a UDP-GlcNAc-4-epimerase is expressed. The nucleic acid sequences encoding said glycosyltransferase enzymes may be cstII (encoding the bifunctional α-2,3 α-2,8-sialyltransferase from C. jejuni), cgtAII from C. jejuni and cgtB from C. jejuni, and the nucleic acid sequence encoding the UDP-GlcNAc-4-epimerase may be gne from C. jejuni or wbpP from P. aeruginosa.

### EXAMPLES

### Example 1: Preparation of the Genetically Engineered Bacterial Strain

The engineered E. coli host strain used in fermentation and the transformed plasmids were constructed in accordance with WO 2007/101862 A1, Fierfort et al. Journal of Biotechnology 134, 261-265 (2008) and Priem et al. Glycobiology 12(4), 234-240 (2002); the strain was engineered from an E. coli K12 strain derivative in which the genes lacA and lacZ as well as the genes nanKETA have been deleted and which has been co-transformed with a plasmid carrying the neuABC genes from Campylobacter jejuni, and a second plasmid carrying the α-2,3-sialyltransferase-encoding nst gene from Neisseria meningitidis (for the production of lyso-GM3 (4a) or with a second plasmid carrying the α-2,3 α-2,8-sialyltransferase-encoding cstII gene from Campylobacter jejuni, for the production of lyso-GD3 (5a).

### Example 2: Synthesis of β-D-Galactopyranosyl-(1→4)-β-D-Glucopyranosyl-(1→1′)-D-Erythro-Sphingosine (1a) (Also Termed Herein as “Lactosylsphingosine”)

Compound 1a was synthesized via a 5-step procedure

### Step 1: Preparation of (2S,3R,4E)-2-N-(1,3-dimethyl-2,4,6(1H,3H,5H)-trioxopyrimidine-5-yilidene)methyl)-octadec-4-ene-1,3-diol

D-erythro-Sphingosine (10 g, 33.4 mmol) is dissolved in methanol (150 mL) at room temperature (r.t.), then DTPM-reagent (7.76 g, 36.8 mmol) is added in one portion. The reaction mixture is stirred at r.t. for 1 h. (After approx. 5 min. crystallization of the product starts.) The slurry is cooled to approx. 5° C., then kept at 5° C. for 2 h. The solid is filtered off (easy filtration on G3), washed with cold methanol (40 mL, 5° C.), then dried in a vacuum oven (30 mbar/40° C./12 h). Yield: 13.5 g (87%).

¹H NMR (400 MHz, CDCl₃): 10.49 (dd, 1H), 8.21 (d, 1H), 5.82 (m, 1H), 5.47 (dd, 1H), 4.37 (m, 1H), 3.94-3.87 (m, 2H), 3.49 (m, 1H), 3.27 and 3.26 (2×s, 3-3H), 3.09 (t, 1H), 2.78 (d, 1H), 2.06 (m, 1H), 1.35-1.24 (m, 23H), 0.87 (t, 3H).

¹³C NMR (400 MHz, CDCl₃): 164.97, 163.24, 152.11, 136.67, 127.34, 91.05, 73.26, 66.01, 61.65, 32.47, 32.06, 29.82, 29.79, 29.73, 29.63, 29.49, 29.34, 29.16, 28.02, 27.30, 22.82, 14.25

### Step 2: Preparation of (2S,3R,4E)-3-O-benzoyl-2-N-((1,3-dimethyl-2,4,6(1H,3H,5H)-trioxopyrimidine-5-ylidene)methyl)-4-octadecene-1-ol

(2S,3R,4E)-2-NH-DTPM-4-octadecene-1,3-diol (10 g, 21.5 mmol) was dissolved in 50 mL dry dichloromethane (50 mL), then pyridine (13 mL) and trityl chloride (7.19 g, 25.8 mmol) were added and the reaction mixture was stirred at room temperature for 20 hours. Benzoyl chloride (3 mL, 25.8 mmol) was added to the reaction mixture and stirred at room temperature for additional 5 hours, then methyl alcohol (0.5 mL) was added. After 3 minutes stirring the reaction mixture was washed twice with 10% hydrochloric acid solution (2×30 mL). Methyl alcohol (8 mL) and p-toluenesulfonic acid monohydrate (0.76 g, 4 mmol) were added to the dichloromethane solution and the mixture was stirred under reflux for 16 hours. After cooling down to room temperature the slurry was washed twice with 5% NaHCO₃ solution (2×20 mL) and once with water (20 mL). The organic phase was dried over MgSO₄, then concentrated in vacuo and the residue was crystallized from isopropanol. Yield: 10.13 g (83%)

¹H NMR (400 MHz, CDCl₃): 10.52 (dd, 1H), 8.24 (d, 1H), 8.02 (m, 2H), 7.59 (m, 1H), 7.47 (m, 2H), 5.95 (m, 1H), 5.65 (m, 1H), 5.53 (dd, 1H), 3.93-3.74 (m, 3H), 3.31 (s, 3H), 3.27 (s, 3H), 2.07 (q, 2H), 1.25 (m, 22H), 0.88 (t, 3H)

¹³C NMR (400 MHz, CDCl₃): 165.68, 164.76, 162.97, 159.81, 151.94, 139.86, 133.62, 129.78, 129.36, 128.56, 122.49, 91.20, 73.38, 65.30, 61.29, 32.43, 31.90, 29.67, 29.64, 29.53, 29.43, 29.33, 29.13, 28.78, 27.83, 27.13, 22.67, 14.10

### Step 3: Preparation of (2S,3R,4E)-3-O-benzoyl-2-N-((1,3-dimethyl-2,4,6(1H,3H,5H)-trioxopyrimidine-5-ylidene)methyl)-4-O-(2,3,4,6-tetra-O-benzoyl-β-D-galactopyranosyl)-β-D-2,3,6-tri-O-benzoyl-glucopyranosyl]-4-octadecene

(2S,3R,4E)-3-O-benzoyl-2-N-((1,3-dimethyl-2,4,6(1H,3H,5H)-trioxopyrimidine-5-ylidene)methyl)-4-octadecene acceptor (5 g, 8.776 mmol) and trichloroacetimidate 4-O-(2,3,4,6-O-tetra-benzoyl-β-D-galactopyranosyl)-2,3,6-tri-O-benzoyl-β-D-glucopyranoside donor (13.87 g, 11.41 mmol) were dissolved in 150 mL dry dichloromethane, then boron trifluoride etherate (0.5 mL, 4.388 mmol) was added and the reaction mixture was stirred at room temperature for 30 minutes. The reaction mixture was quenched by adding 5% NaHCO₃ solution (70 mL) and stirred at room temperature for 1 hour. The phases were separated, the organic phase was concentrated in vacuo and the residue was purified by column chromatography. Yield: 13.4 g (94%)

¹H NMR (400 MHz, CDCl₃): 10.35 (dd, 1H), 8.18 (d, 1H), 8.05-7.15 (m, 40H), 5.72-5.85 (m, 4H), 5.49-5.58 (m, 2H), 5.33-5.40 (m, 2H), 4.85 (d, 1H), 4.77 (d, 1H), 4.53 (m, 2H), 4.27 (m, 1H), 3.84-3.99 (m, 5H), 3.67-3.78 (m, 2H), 3.22 (s, 3H), 3.17 (s, 3H), 1.27 (m, 24H), 0.90 (t, 3H)

¹³C NMR (400 MHz, CDCl₃): 165.63, 165.49, 165.36, 165.28, 165.16, 164.98, 164.73, 164.66, 162.46, 159.50, 151.90, 139.77, 133.49, 133.36, 133.13, 129.96, 129.83, 129.77, 129.72, 129.63, 129.51, 129.45, 129.36, 129.27, 129.10, 128.84, 128.64, 128.61, 128.55, 128.24, 121.64, 100.94, 100.43, 91.50, 75.57, 73.39, 73.26, 72.62, 71.74, 71.33, 71.25, 69.82, 67.44, 67.16, 62.79, 62.01, 60.93, 32.36, 31.89, 29.67, 29.63, 29.52, 29.37, 29.33, 29.11, 28.69, 27.66, 26.94, 22.66, 14.10

### Step 4: Preparation of (2S,3R,4E)-2-N-((1,3-dimethyl-2,4,6(1H,3H,5H)-trioxopyrimidine-5-ylidene)methyl)-1-O-[4-O-(β-D-galactopyranosyl)-β-D-glucopyranosyl]-3-hydroxy-4-octadecene

(2S,3R,4E)-3-O-benzoyl-2-N-((1,3-dimethyl-2,4,6(1H,3H,5H)-trioxopyrimidine-5-ylidene)methyl)-1-O-[2,3,6-tri-O-benzoyl-4-O-(2,3,4,6-tetra-O-benzoyl-β-D-galactopyranosyl)-3-D-glucopyranoside]-4-octadecene (10 g, 6.16 mmol) were added to dry methanol (200 mL) and 25% NaOMe in methanol (0.14 mL, 0.616 mmol) was added. The reaction mixture was stirred at room temperature for 2 days. The product crystallized out of the reaction mixture. The crystallization slurry was cooled down to 0-5° C., stirred at 0-5° C. for 2 hours. The solid was filtered off, washed with cold methanol (2×10 mL), then dried in a vacuum oven (30 mbar/50° C./12 h). Yield: 4.44 g (91%)

¹H NMR (400 MHz, DMSO): 8.19 (s, 1H), 5.63 (m, 1H), 5.36 (dd, 1H), 4.21 (m, 3H), 3.94 (m, 1H), 3.78 (m, 2H), 3.70-3.54 (m, 6H), 3.41-3.31 (m, 7H), 3.14 (m, 11H), 2.50 (s, 1H), 1.95 (m, 2H), 1.17 (m, 24H), 0.79 (t, 3H)

¹³C NMR (400 MHz, DMSO): 166.18, 164.74, 161.50, 153.59, 136.30, 129.74, 105.34, 104.38, 91.65, 80.66, 77.21, 76.73, 76.46, 74.96, 74.72, 72.58, 70.28, 69.02, 65.95, 62.49, 62.03, 33.49, 33.15, 30.91, 30.89, 30.86, 30.84, 30.81, 30.78, 30.56, 30.41, 30.32, 28.37, 27.74, 23.85, 14.76

### Step 5: Preparation of Compound 1a

(2S,3R,4E)-2-((1,3-dimethyl-2,4,6(1H,3H,5H)-trioxopyrimidine-5-ylidene)methyl)-1-O-[4-O-(β-D-galactopyranosyl)-3-D-glucopyranoside]-3-hydroxy-4-octadecene (2 g, 2.532 mmol) and N,N-dimethyl-1,3-propanediamine (0.95 mL, 7.596 mmol) were added to 10 mL dry methanol. The reaction mixture was stirred at 60° C. for 3 hours, then at room temperature for an additional 12 hours. The product crystallized out of the reaction mixture. The crystallization slurry was cooled down to 0-5° C., stirred at 0-5° C. for 1 hour. The solid was filtered off, washed with cold methanol (2×4 mL), then dried in a vacuum oven (30 mbar/50° C./12 h). Yield: 1.43 g (90%)

¹H NMR (600 MHz, DMSO): 5.57 (ddd, 1H), 5.48 (dd, 1H), 5.28 (s, 2H), 5.08 (d, 1H), 4.78 (d, 1H) 4.67 (s, 1H), 4.66 (s, 1H), 4.65 (t, 1H), 4.56 (t, 1H), 4.51 (d, 1H), 4.20 (d, 1H), 4.17 (d, 1H), 3.8-3.73 (m, 3H), 3.61 (t, 1H), 3.60-3.52 (m, 4H), 3.48-3.45 (m, 2H), 3.32-3.28 (m, 5H), 3.02 (t, 1H), 2.76 (m, 1H), 1.99 (m, 2H), 1.40-1.15 (m, 24H), 0.85 (t, 3H)

¹³C NMR (600 MHz, DMSO): 131.25, 131.05, 103.82, 102.676 80.67, 75.49, 74.81, 73.21, 73.09, 72.77, 71.26, 70.50, 68.09, 60.43, 60.35, 55.04, 31.75, 31.26, 29.02, 28.91, 28.84, 28.81, 28.67, 28.59, 22.06, 13.92

### Example 3: Synthesis of β-D-galactopyranosyl-(1→4)-β-D-glucopyranosyl-(1→1′)-D-erythro-N-acetyl-sphingosine (1b) (Also Termed Herein as “N-acetyl-lactosylsphingosine”)

To a round bottom flask containing acetic acid (60 mL, 33.6 mmol) and triethylamine (4.9 ml, 35.2 eq) in tetrahydrofuran (200 mL), Lactosylsphingosine (1a) (20 g, 32 mmol) is added in portions, and the reaction mixture heated to 60° C. for 20 h. Upon confirmation of the conversion by TLC, the reaction mixture is then cooled slowly to 4° C. and stirring maintained by further 4 h.

The solid precipitate is then filtered and washed with cold THF. The resulting N-acetyl-lactosylsphingosine (1b) is then purified by silica column chromatography (chloroform/methanol/water (65:25:4)), providing an off-white solid (14.9 g, 69.8%).

### Example 4: General Fermentation Procedure

The culture was carried out in a 2 l fermenter containing 1 liter of minimal medium containing ammonium phosphate 87 mM, potassium phosphate 51 mM, TMS-A, Citric Acid 5.2 mM, potassium hydroxide 45 mM, sodium hydroxide 25 mM, magnesium sulphate 2.5 mMb as well as Glucose 15.9 g/L and Glycerol 2.4 g/L as initial carbon source. The growth phase started with the inoculation (2% inoculum). The temperature was kept at around 33° C. and the pH regulated at 6.8 with aqueous NH₄₀H solution. The oxygen was kept at 40% with an air flow between 0.5 to 3 L/min until cells were adapted to the glycerol in the medium. When all initial carbon source was depleted, the fed-batch phase was initiated, the exogenous precursor 1a or 1b (16 mM) and the inducer IPTG (1-2 ml of a 50 ng/ml solution) were added to the culture and the temperature was decreased to 28° C. The fed-batch was realized using a 750 g/L aqueous glycerol solution, with a high substrate feeding rate of ≈4.5 g/h of glycerol for 1 l of culture. The maximal production yield for compounds 4a and 5a was observed after 72 h.

### Example 5—General Purification Procedure

The fermentation broth was ultrafiltered (5-30 kDa membrane) at 25° C. until the total volume was concentrated to half and the UF permeate was collected. The UF retentate was then washed with purified water (4 to 5-fold volumes relative to the ultrafiltered broth volume) until all compound of interest was extracted to the permeate. The combined UF permeates were then subjected to nanofiltration (300-500 Da membrane) at 30 bar and 15° C. until the retentate reached a concentration 20 to 30-fold higher than the initial solution.

The NF retentate was subjected to standard chromatographic techniques to afford the final compounds.

### Example 6: α-N-acetylneuroaminoyl-(2→3)-β-D-galactopyranosyl-(1→4)-β-D-glucopyranosyl-(1→1′)-D-erythro-sphingosine (Lyso-GM3, 4a)

Compound 4a was obtained following the general fermentation and purification procedures described in examples 4 and 5.

¹H NMR (400 MHz, MeOD) δ 5.88 (dq, J=14.5, 7.6, 7.2 Hz, 1H), 5.51 (dd, J=15.3, 6.7 Hz, 1H), 4.44 (d, J=7.8 Hz, 1H), 4.39 (d, J=7.8 Hz, 1H), 4.33 (t, J=5.7 Hz, 1H), 4.07 (dd, J=9.7, 3.1 Hz, 1H), 4.02-3.20 (m, 26H), 2.88 (dd, J=12.4, 3.8 Hz, 1H), 2.17-2.04 (m, 6H), 1.75 (t, J=11.5 Hz, 1H), 1.45 (p, J=7.0 Hz, 2H), 1.33 (d, J=8.6 Hz, 11H), 1.20 (t, J=7.0 Hz, 1H), 0.92 (t, J=6.7 Hz, 3H).

¹³C NMR (101 MHz, MeOD) δ 175.60, 174.94, 136.67, 128.37, 105.10, 103.80, 101.09, 80.53, 77.72, 77.14, 76.59, 76.22, 74.98, 74.50, 73.03, 70.97, 70.82, 70.13, 69.28, 69.04, 67.32, 64.71, 62.77, 61.62, 58.34, 56.74, 53.98, 49.67, 49.46, 49.24, 49.03, 48.82, 48.60, 48.39, 42.10, 33.42, 33.11, 30.84, 30.80, 30.68, 30.52, 30.45, 30.23, 23.77, 22.64, 14.50.

### Example 7: α-N-acetylneuroaminoyl-(2→8)-α-5-N-acetylneuroaminoyl-(2→3)-β-D-galactopyranosyl-(1→4)-β-D-glucopyranosyl-(1→1′)-D-erythro-sphingosine (Lyso-GD3, 5a)

Compound 5a was obtained following the general fermentation and purification procedures described in examples 4 and 5. MS: 1206.3 Da [M+H]⁺.

### Example 8: α-N-acetylneuroaminoyl-(2→3)-β-D-galactopyranosyl-(1→4)-β-D-glucopyranosyl-(1→1′)-D-erythro-N-acetyl-sphingosine (7a)

Compound 7a was obtained following the general fermentation and purification procedures described in examples 4 and 5. MS: 979.5 Da [M+Na]⁺.

1. Method for producing a glycosylated sphingoid base of interest or an analogue thereof, the method comprising the steps of:
a) Providing an exogenous precursor and a genetically modified cell, wherein one or more glycosylation reactions can be performed on the exogenous precursor or on the glycosylated derivative thereof in the genetically modified cell, the genetically modified cell comprising one or more nucleic acid sequences encoding one or more glycosyltransferases, and wherein the exogenous precursor is a compound of General Formula I
Y—X—R General Formula I,
wherein
Y is a glycosyl moiety,
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 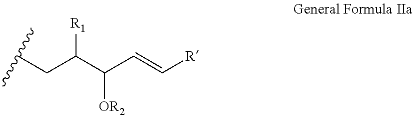
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 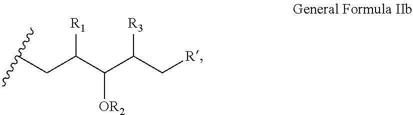
wherein R′, R₁, R₂, are as defined in General Formula IIa, and
R₃ is H, OH or OR₆, wherein R₆ is selected from substituted or unsubstituted alkyl or substituted or unsubstituted acyl;
b) Culturing said genetically modified cell in a culture medium comprising said exogenous precursor, whereby
i. the exogenous precursor is internalized by the cell, and
ii. one or more glycosylation reactions are performed on the internalized exogenous precursor or on a glycosylated derivative thereof by the one or more glycosyltransferases, to form the glycosylated sphingoid base of interest,
c) Optionally isolating the glycosylated sphingoid base of interest or the analogue thereof characterized by General Formula III from the genetically modified cell and/or from the culture medium.

2. The method according to claim 1, wherein the genetically modified cell is a yeast cell or a bacterial cell.

3. The method according to claim 1, wherein the one or more glycosyltransferase enzymes comprise one or more sialyltransferases and/or one or more fucosyltransferases, especially one or more sialyltransferases.

4. The method according to claim 1, wherein the one or more glycosyltransferase enzymes are selected from the group consisting of β-1,3-N-acetylglucosaminyltransferase, β-1,6-N-acetylglucosaminyltransferase, β-1,3-galactosyltransferase, β-1,4-galactosyltransferase, β-1,4-N-acetylgalactosaminyltransferase, β-1,3-N-acetylgalactosaminyltransferase, β-1,3-glucoronosyltransferase, α-2,3-sialyltransferase, α-2,6-sialyltransferase, α-2,8-sialyltransferase, α-1,2-fucosyltransferase, α-1,3-fucosyltransferase, α-1,4-fucosyltransferase, α-1,4-galactosyltransferase, α-1,3-galactosyltransferase or a combination thereof.

5. The method according to claim 1, wherein Y of General Formula I is a monosaccharide moiety, a disaccharide moiety or a trisaccharide moiety.

6. The method according to claim 1, wherein the exogenous precursor is a compound of General Formula Ia: 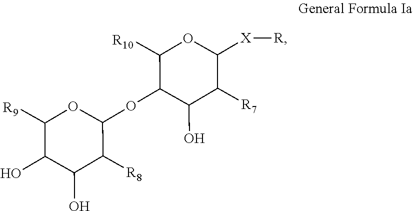
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 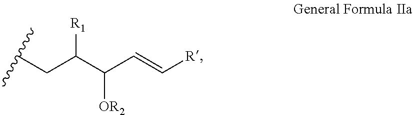
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 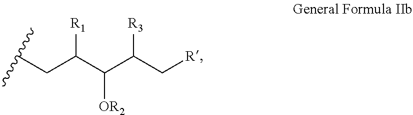
wherein R′, R₁, R₂ are as defined in General Formula IIa, and
R₃ is H, OH or OR₆, wherein R₆ is selected from substituted or unsubstituted alkyl or substituted or unsubstituted acyl
R₇ and R₈ are independently selected from the group consisting of OH, NH₂ and NH-acyl, and
R₉ and R₁₀ are independently selected from the group consisting of —CH₂—OH and an C₁₋₆ alkyl.

7. The method according to claim 1, wherein the exogenous precursor is a compound of General Formula Ib: 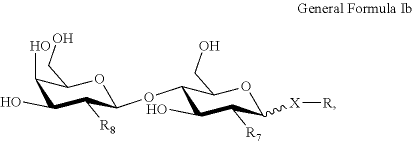
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 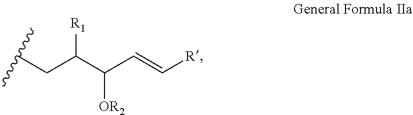
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 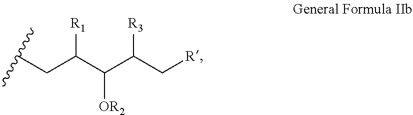
wherein R′, R₁, R₂ are as defined in General Formula IIa, and
R₃ is H, OH or OR₆, wherein R₆ is selected from substituted or unsubstituted alkyl or substituted or unsubstituted acyl and
R₇ and R₈ are independently selected from the group consisting of OH, NH₂ and NH-acyl.

8. The method according to claim 1, wherein the exogenous precursor is a compound of General Formula Ic: 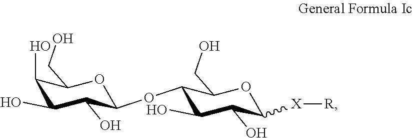
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 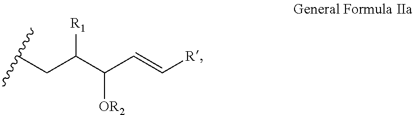
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 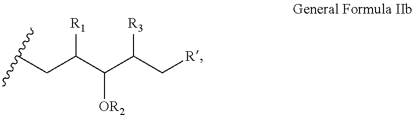
wherein R′, R₁, R₂ are as defined in General Formula IIa, and
R₃ is H, OH or OR₆, wherein R₆ is selected from substituted or unsubstituted alkyl or substituted or unsubstituted acyl
and
wherein glycosidic bond [symbol] is a glycosidic bond.

9. The method according to claim 1, wherein the genetically modified cell has no β-galactosidase activity.

10. The method according to claim 1, wherein the glycosylated sphingoid base of interest is a compound of General Formula IIIa: 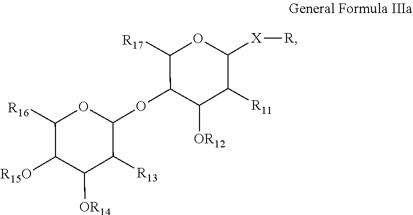
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 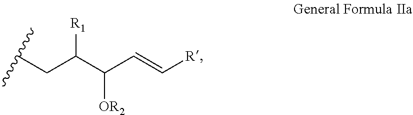
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 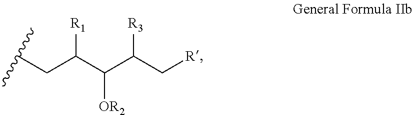
wherein R′, R₁, R₂ are as defined in General Formula IIa, and
R₃ is H, OH or OR₆, wherein R₆ is selected from substituted or unsubstituted alkyl or substituted or unsubstituted acyl;
R₁₁ and R₁₃ are independently selected from the group consisting of OH, NH₂, NH-acyl and O-glycoside,
R₁₂, R₁₄ and R₁₅ are independently selected from the group consisting of hydrogen and a glycosyl moiety,
and
R₁₆ and R₁₇ are independently selected from the group consisting of CH₂—OH, CH₂O-glycoside and C₁₋₆ alkyl.

11. The method according to claim 1, wherein the glycosylated sphingoid base of interest is a compound of General Formula IIIb: 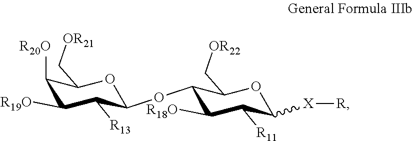
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 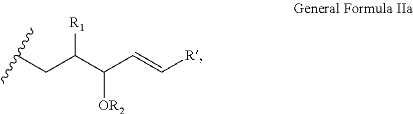
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 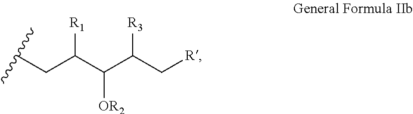
wherein R′, R₁, R₂ are as defined in General Formula IIa, and
R₃ is H, OH or OR₆, wherein R₆ is selected from substituted or unsubstituted alkyl or substituted or unsubstituted acyl;
R₁₁ and R₁₃ are independently selected from the group consisting of OH, NH₂, NH-acyl and O-glycoside,
and
R₁₈ R₁₉ R₂₀ R₂₁, R₂₂ are independently selected from the group consisting of hydrogen or a glycosyl moiety.

12. The method according to claim 1, wherein the glycosylated sphingoid base of interest is a compound of General Formula IIIc: 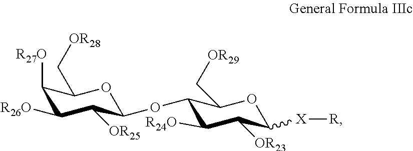
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 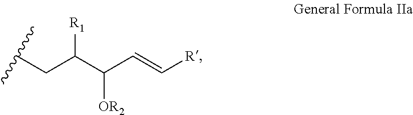
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 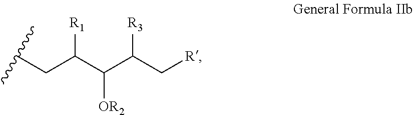
wherein R′, R₁, R₂ are as defined in General Formula IIa, and
R₃ is H, OH or OR₆, wherein R₆ is selected from substituted or unsubstituted alkyl or substituted or unsubstituted acyl
and
R₂₃, R₂₄, R₂₅, R₂₆, R₂₇, R₂₈, R₂₉ are independently hydrogen or a glycosyl moiety.

13. The method according to claim 8, wherein the glycosyltransferase enzyme is a α-2,3-sialyltransferase and the produced glycosylated sphingoid base of interest is compound of General Formula IV or a salt thereof: 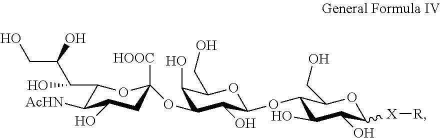
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 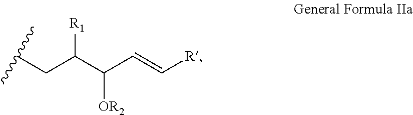
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 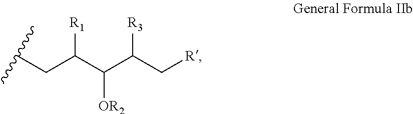
wherein R′, R₁, R₂ are as defined in General Formula IIa, and
R₃ is H, OH or OR₆, wherein R₆ is selected from substituted or unsubstituted alkyl or substituted or unsubstituted acyl
and
glycosidic bond [symbol] is a glycosidic bond.

14. The method according to claim 8, wherein the glycosyltransferase enzymes are α-2,8-sialyltransferase and α-2,3-sialyltransferase and the produced glycosylated sphingoid base of interest is compound of General Formula V or a salt thereof: 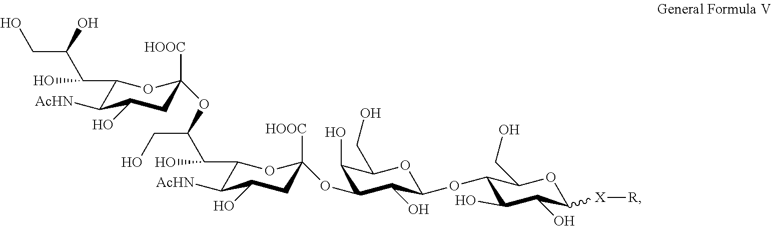
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 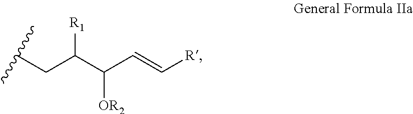
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 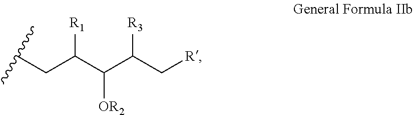
wherein R′, R₁, R₂ are as defined in General Formula IIa, and
R₃ is H, OH or OR₆, wherein R₆ is selected from substituted or unsubstituted alkyl or substituted or unsubstituted acyl;
and
glycosidic bond [symbol] is a glycosidic bond.

15. The method according to claim 8, wherein the glycosyltransferase enzymes are β-1,4-N-acetylgalactosaminyltransferase, β-1,3-galactosyltransferase and α-2,3-sialyltransferase, and the produced glycosylated sphingoid base of interest is compound of General Formula VI or a salt thereof: 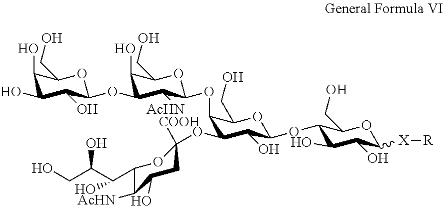
wherein
X is O, S, NH or CH₂, linking Y to R by an O-, S-, N- or C-glycosidic linkage, respectively, wherein the glycosidic linkage is a glycosidic linkage,
and R is a group of General Formula IIa or General Formula IIb: 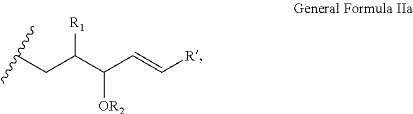
wherein
R′ is H, aryl or an alkyl chain having 1-43 carbon atoms, which may be a straight chain or branched, and/or which may be saturated or contain one or more double and/or triple bonds, and/or which may contain one or more functional groups,
R₁ is N₃ or NR₄R₅, wherein R₄ and R₅ are independently selected from H, substituted or unsubstituted alkyl, substituted or unsubstituted aryl, substituted or unsubstituted vinyl, substituted or unsubstituted acyl, or wherein R₄ and R₅ form a cyclic structure,
R₂ is substituted or unsubstituted alkyl or substituted or unsubstituted acyl; 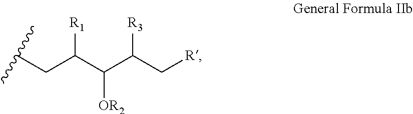
wherein R′, R₁, R₂ are as defined in General Formula IIa, and
R₃ is H, OH or OR₆, wherein R₆ is selected from substituted or unsubstituted alkyl or substituted or unsubstituted acyl;
and
glycosidic bond [symbol] is a glycosidic bond.

16. The method according to claim 1, wherein X is O and/or R is a group of formula 2a or of formula 2b:

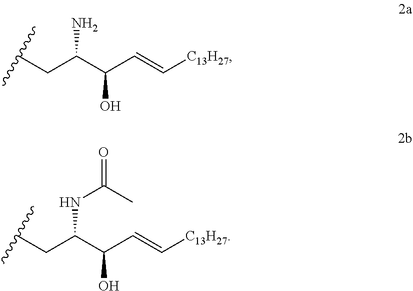

17. Compound of General Formula VII: 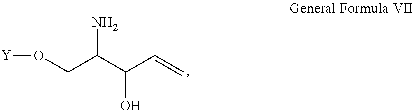
wherein
Y is a glycosyl moiety selected from:
Galβ1-3GalNAcβ1-4(Neu5Acα2-3)Galβ1-4Glc1-,
Neu5Acα2-3Galβ1-3GalNAcβ1-4Galβ1-4Glc1-,
GalNAcβ1-4(Neu5Acα2-3)Galβ1-4Glc1-,
Neu5Acα2-3Galβ1-4Glc1-,
Neu5Acα2-3Galβ1-,
Neu5Acα2-3Galβ1-3GalNAcβ1-4(Neu5Acα2-3)Galβ1-4Glc1-,
Galβ1-3GalNAcβ1-4(Neu5Acα2-8Neu5Acα2-3)Galβ1-4Glc1-,
Neu5Acα2-8Neu5Acα2-3Galβ1-3GalNAcβ1-4Galβ1-4Glc1-,
Neu5Acα2-3Galβ1-3(Neu5Acα2-6)GalNAcβ1-4Galβ1-4Glc1-,
GalNAcβ1-4(Neu5Acα2-8Neu5Acα2-3)Galβ1-4Glc-,
Neu5Acα2-8Neu5Acα2-3Galβ1-4Glc1-,
Neu5Acα2-8Neu5Acα2-3Galβ1-3GalNAcβ1-4(Neu5Acα2-3)Galβ1-4Glc1-,
Neu5Acα2-3Galβ1-3GalNAcβ1-4(Neu5Acα2-8Neu5Acα2-3)Galβ1-4Glc1-,
Galβ1-3GalNAcβ1-4(Neu5Acα2-8Neu5Acα2-8 Neu5Acα2-3)Galβ1-4Glc1-,
Neu5Acα2-3Galβ1-3(Neu5Acα2-6)GalNAcβ1-4(Neu5Acα2-3)Galβ1-4Glc1-,
GalNAcβ1-4(Neu5Acα2-8Neu5Acα2-8 Neu5Acα2-3)Galβ1-4Glc1-,
Neu5Acα2-8Neu5Acα2-8 Neu5Acα2-3Galβ1-4Glc1-,
Neu5Acα2-8Neu5Acα2-3Galβ1-3GalNAcβ1-4(Neu5Acα2-8Neu5Acα2-3)Galβ1-4Glc1-,
Neu5Acα2-3Galβ1-3(Neu5Acα2-6)GalNAcβ1-4(Neu5Acα2-8Neu5Acα2-3)Galβ1-4Glc1-,
Neu5Acα2-3Galβ1-3GalNAcβ1-4(Neu5Acα2-8Neu5Acα2-8Neu5Acα2-3) Galβ1-4Glc1-,
Galα1-4Galβ1-4Glc1-,
Galα1-3Galβ1-4Glc1-,
GalNAcβ1-3Galα1-4Galβ1-4Glc1-,
GalNAcβ1-3Galα1-3Galβ1-4Glc1-,
Galβ1-3GalNAcβ1-3Galα1-4Galβ1-4Glc1-,
Neu5Acα2-3Galβ1-3GalNAcβ1-3Galα1-4Galβ1-4Glc1-,
Neu5Acα2-3Galβ1-3(Neu5Acα2-6)GalNAcβ1-3Galα1-4Galβ1-4Glc1-,
GalNAcα1-3GalNAcβ1-3Galα1-4Galβ1-4Glc1-,
GalNAcβ1-3GalNAcβ1-3Galα1-4Gal P1-4Glc1-,
GalNAcα1-3GalNAcβ1-3(Galβ1-3GalNAcβ1-4)Galα1-4Galβ1-4Glc1-,
Galβ1-3GalNAcβ1-3Galα1-4Galβ1-4Glc1-,
Fucα1-2Galβ1-3GalNAcβ1-3Galα1-4Galβ1-4Glc1-,
GalNAcα1-3(Fucα1-2)Galβ1-3GalNAcβ1-3Galα1-4Galβ1-4Glc1-,
Galα1-3(Fucα1-2)Galβ1-3GalNAcβ1-3Galα1-4Galβ1-4Glc1-,
Galβ1-4(Fucα1-3)GlcNAcβ1-6(Galβ1-3)GalNAcβ1-3Galα1-4Galβ1-4Glc1-,
Galβ1-4GlcNAcβ1-3Galβ1-4Glc1-,
GlcNAcβ1-3Galβ1-4GlcNAcβ1-3Galβ1-4Glc1-,
Galβ1-4GlcNAcβ1-3Galβ1-4GlcNAcβ1-3Galβ1-4Glc1-,
Galβ1-4(Fucα1-3)GlcNAcβ1-3Galβ1-4Glc1-,
Galβ1-4(Fucα1-3)GlcNAcβ1-3Galβ1-4GlcNAcβ1-3Galβ1-4Glc1-,
Galβ1-4(Fucα1-3)GlcNAcβ1-3Galβ1-4(Fucα1-3)GlcNAcβ1-3Galβ1-4Glc1-,
Galβ1-4GlcNAcβ1-3(Galβ1-4GlcNAcβ1-6)Galβ1-4GlcNAcβ1-3Galβ1-4Glc1-,
Fucα1-2Galβ1-4GlcNAcβ1-3Galβ1-4Glc1-,
GalNAcβ1-3(Fucα1-2)Galβ1-4GlcNAcβ1-3Galβ1-4Glc1-,
GalNAcα1-3(Fucα1-2)Galβ1-4GlcNAcβ1-3Galβ1-4Glc1-,
Galα1-3(Fucα1-2)Galβ1-4GlcNAcβ1-3Galβ1-4Glc1-,
Fucα1-2Galβ1-3GalNAcα1-3(Fucα1-2)Galβ1-4GlcNAcβ1-3Galβ1-4Glc1-,
Fucα1-2Galβ1-4(Fucα1-3)GlcNAcβ1-3Galβ1-4Glc1-,
Fucα1-2Galβ1-4(Fucα1-3)GlcNAcβ1-3Galβ1-4GlcNAcβ1-3Galβ1-4Glc1-,
Galβ1-3(Fucα1-4)GlcNAcβ1-3Galβ1-4(Fucα1-3)GlcNAcβ1-3Galβ1-4Glc1-,
Galβ1-3Galβ1-4GlcNAcβ1-3Galβ1-4Glc1-,
GalNAcβ1-3Galβ1-4GlcNAcβ1-3Galβ1-4Glc1-,
Galα1-3Galβ1-4GlcNAcβ1-3(GalNAcβ1-4)Galβ1-4Glc1-,
GlcNAcβ1-3Galβ1-4(Fucα1-3)GlcNAcβ1-3Galβ1-4Glc1-,
Galα1-3Galβ1-4(Fucα1-3)GlcNAcβ1-3Galβ1-4Glc1-,
Galβ1-4Galβ1-4GlcNAcβ1-3Galβ1-4Glc1-,
GalNAcβ1-3Galα1-3Galβ1-4GlcNAcβ1-3Galβ1-4Glc1-,
Galβ1-3GlcNAcβ1-3Galβ1-4Glc1-,
Galβ1-4GlcNAcβ1-3Galβ1-4Glc1-,
Galβ1-3GlcNAcβ1-3(Galβ1-4GlcNAcβ1-6)Galβ1-4Glc1-,
Galβ1-4GlcNAcβ1-3(Galβ1-4GlucNAcβ1-6)Galβ1-4Glc1-,
Fucα1-2Galβ1-4Glc1-,
Galβ1-4(Fucα1-3)Glc1-,
Fucα1-2Galβ1-4(Fucα1-3)Glc1-,
Fucα1-2Galβ1-3GlcNAcβ1-3Galβ1-4Glc1-,
Galβ1-3(Fucα1-4)GlcNAcβ1-3Galβ1-4Glc1-,
Galβ1-3(Fucα1-3)GlcNAcβ1-3Galβ1-4Glc1-,
Galβ1-3GlcNAcβ1-3Galβ1-4(Fucα1-3)Glc1-,
Fucα1-2Galβ1-3(Fucα1-4)GlcNacβ1-3Galβ1-4Glc1-,
Neu5Acα2-3Galβ1-4Glc1-,
Neu5Acα2-6Galβ1-4Glc1-,
Neu5Acα2-3Galβ1-4(Fucα1,3)Glc1-,
Neu5Acα2-3Galβ1-3GlcNAcβ1-3Galβ1-4Glc1-,
Galβ1-3(Neu5Acα2-6)GlcNAcβ1-3Galβ1-4Glc1-,
Neu5Acα2-6Galβ1-3GlcNAcβ1-3Galβ1-4Glc1-,
Neu5Acα2-3Galβ1-3(Neu5Acα2-6)GlcNAcβ1-3Galβ1-4Glc1-,
Galβ1-3Galβ1-4Glc1-,
Galβ1-4Galβ1-4Glc1-,
Galβ1-6Galβ1-4Glc1-,
Galβ1-3GalNAcβ1-4Galβ1-4Glc1-,
Fucα1-2Galβ1-3(Fucα1-4)GlcNAcβ1-3Galβ1-4Glc1-.

18. (canceled)

19. (canceled)

20. (canceled)

21. (canceled)
